(19) United States
(12) Patent Application Publication
(10) Pub. No.: US 2026/0256476 A1
(43) Pub. Date: Sep. 3, 2026

(54) CLIP CARTRIDGE AND METHOD OF RELOADING END EFFECTOR

(71) Applicant: Cilag GmbH International, Zug (CH)

(72) Inventors: Disha V. Estera, Cincinnati, OH (US); Kris E. Kallenberger, Cincinnati, OH (US); Scott D. Harrington, Kirkwood, MO (US); Michael J. Stokes, Cincinnati, OH (US)

(21) Appl. No.: 19/067,556

(22) Filed: Feb. 28, 2025

(51) Int. Cl. A61B 17/128; A61B 17/34

(52) U.S. Cl. CPC A61B 17/1285; A61B 17/3468

(57) ABSTRACT

An apparatus includes a proximal instrument coupler configured to releasably attach to an end effector of a surgical instrument. The apparatus also includes a distal clip magazine extending distally from the proximal instrument coupler along a longitudinal axis. The distal clip magazine is sized and configured to pass through a working channel of a trocar into a patient's body. The distal clip magazine includes a longitudinal array of clip receptacles. Each clip receptacle of the longitudinal array of clip receptacles is configured to selectively retain a respective surgical clip.

### BACKGROUND

A variety of surgical instruments include an end effector for use in conventional medical treatments and procedures conducted by a medical professional operator, as well as applications in robotically assisted surgeries. Such surgical instruments may be directly gripped and manipulated by a surgeon or incorporated into robotically assisted surgery. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletal gloves, master manipulators, or the like), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probe, etc. Each of these structures performs functions for the surgeon, for example, cutting tissue, coagulating tissue, holding or driving a needle, grasping a blood vessel, dissecting tissue, cauterizing tissue, and/or other functions.

Surgical clips may be used for a number of surgical procedures. In endoscopic or laparoscopic surgical procedures, access to the surgical site may be achieved through a trocar inserted through a small entrance incision in the skin. The trocar port allows the surgeon to insert a number of different surgical instruments therethrough and to perform surgical procedures within the patient in a minimally invasive manner.

During some surgical procedures, the surgeon may wish to terminate the flow of blood or another fluid through one or more vessels. In some such instances, the surgeon may apply a surgical clip to a blood vessel or another duct to prevent the flow of blood or other bodily fluids therethrough during the procedure. An endoscopic surgical clip applier is capable of applying a singular surgical clip or multiple surgical clips during a minimally invasive entry to the body cavity. For instance, an endoscopic surgical clip applier is capable of ligating a blood vessel by clamping a surgical clip about the blood vessel to thereby prevent blood flow through the vessel. Such clips may be fabricated from a malleable biocompatible material and may be compressed over a vessel. Alternatively, such clips may be fabricated from a resilient biocompatible material and may be released to resiliently clamp the vessel.

Examples of surgical clips are represented by the LIGACLIP® series of surgical clips by Ethicon Endo-Surgery, Inc. of Cincinnati, Ohio. One example of a surgical clip applier is the LIGAMAX™ 5 by Ethicon Endo-Surgery, Inc. of Cincinnati, Ohio. Other examples of surgical clip appliers are represented by the LIGACLIP® series of surgical clip appliers by Ethicon Endo-Surgery, Inc. of Cincinnati, Ohio. Still further examples of surgical clip appliers and surgical clips are disclosed in U.S. Pat. No. 5,163,945, entitled “Surgical Clip Applier,” issued Nov. 17, 1992, the disclosure of which is incorporated by reference herein; U.S. Pat. No. 5,342,373, entitled “Sterile Clips and Instrument for their Placement,” issued Aug. 30, 1994, the disclosure of which is incorporated by reference herein; U.S. Pat. No. 5,431,668, entitled “Ligating Clip Applier,” issued Jul. 11, 1995, the disclosure of which is incorporated by reference herein; U.S. Pat. No. 5,445,167, entitled “Methods of Applying Surgical Clips and Suture Tie Devices to Bodily Tissue During Endoscopic Procedures,” issued Aug. 29, 1995, the disclosure of which is incorporated by reference herein; U.S. Pat. No. 5,601,573, entitled “Sterile Occlusion Fasteners and Instruments and Methods for Their Placement,” issued Feb. 11, 1997, the disclosure of which is incorporated by reference herein; U.S. Pat. No. 5,951,574, entitled “Multiple Clip Applier Having a Split Feeding Mechanism,” issued Sep. 14, 1999, the disclosure of which is incorporated by reference herein; U.S. Pat. No. 7,261,724, entitled “Surgical Clip Advancement Mechanism,” issued Aug. 28, 2007, the disclosure of which is incorporated by reference herein; U.S. Pat. No. 7,686,820, entitled “Surgical Clip Applier Ratchet Mechanism,” issued Mar. 30, 2010, the disclosure of which is incorporated by reference herein; U.S. Pat. No. 7,699,860, entitled “Surgical Clip,” issued Apr. 20, 2010, the disclosure of which is incorporated by reference herein; U.S. Pat. No. 7,731,724, entitled “Surgical Clip Advancement and Alignment Mechanism,” issued Jun. 8, 2010, the disclosure of which is incorporated by reference herein; U.S. Pat. No. 8,038,686, entitled “Clip Applier Configured to Prevent Clip Fallout,” issued Oct. 18, 2011, the disclosure of which is incorporated by reference herein; and U.S. Pat. No. 8,262,679, entitled “Clip Advancer,” issued Sep. 11, 2012, the disclosure of which is incorporated by reference herein.

In some instances, the surgeon may desire to load a surgical clip applier with one or more surgical clips while the surgical clip applier is positioned within a patient. For example, the surgeon may employ the surgical clip applier to apply a first surgical clip to a vessel within the patient, and may then desire to re-load the surgical clip applier with a second surgical clip for application to the same or a different vessel within the patient; and may desire to perform such re-loading without having to first retract the surgical clip applier out of the patient. The clip cartridge and method of reloading an end effector of the present disclosure seek to provide intracorporeal surgical clip loading.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first illustrative robotic system configured for a laparoscopic procedure;

FIG. 2 depicts a perspective view of a second illustrative robotic system;

FIG. 3 depicts an end elevational view of the robotic system of FIG. 2;

FIG. 4 depicts an end elevational view of the robotic system of FIG. 2 including an illustrative pair of robotic arms;

FIG. 5 depicts a partially exploded perspective view of the robotic arm of FIG. 4 having an instrument driver and a first illustrative surgical instrument;

FIG. 6A depicts a side elevational view of the surgical instrument of FIG. 5 in a retracted position;

FIG. 6B depicts a side elevational view the surgical instrument of FIG. 5 in an extended position;

FIG. 7A depicts an enlarged perspective view of a second illustrative surgical instrument, more particularly a needle driver instrument;

FIG. 7B depicts another enlarged perspective view of the needle driver instrument of FIG. 7A, with a distal clevis of the needle driver instrument illustrated as transparent to show certain internal features thereof;

FIG. 7C depicts a side elevational view of the needle driver instrument of FIG. 7A;

FIG. 7D depicts another side elevational view of the needle driver instrument of FIG. 7A;

FIG. 7E depicts a top plan view of a proximal clevis of the needle driver instrument of FIG. 7A;

FIG. 8 depicts a perspective view of an example of a surgical system including a forceps, a surgical clip applier, a plurality of surgical clips, and a surgical clip cartridge;

FIG. 9 depicts an exploded perspective view of the surgical system of FIG. 8;

FIG. 10 depicts a top plan view of the surgical clip cartridge of FIG. 8;

FIG. 11 depicts a side elevational view of the surgical clip cartridge of FIG. 8;

FIG. 12 depicts a cross-sectional view of the surgical clip cartridge of FIG. 8, taken along line 12-12 in FIG. 10;

FIG. 13 depicts a cross-sectional view of the surgical clip cartridge of FIG. 8, taken along line 13-13 in FIG. 10;

FIG. 14A depicts a side elevational view of the surgical clip cartridge of FIG. 8, showing the surgical clips of FIG. 8 being loaded into corresponding clip receptacles of the surgical clip cartridge;

FIG. 14B depicts a side elevational view of the surgical clip cartridge of FIG. 8, showing the surgical clip cartridge loaded with the surgical clips of FIG. 8 and aligned with the forceps of FIG. 8;

FIG. 14C depicts a side elevational view of the surgical clip cartridge of FIG. 8, showing the surgical clip cartridge loaded with the surgical clips of FIG. 8 and attached to the forceps of FIG. 8;

FIG. 14D depicts a side elevational view of the surgical clip cartridge of FIG. 8, showing the surgical clip cartridge loaded with the surgical clips of FIG. 8, attached to the forceps of FIG. 8, and inserted through a working channel of a trocar and into a patient's body;

FIG. 14E depicts a side elevational view of the surgical clip cartridge of FIG. 8, showing the surgical clip cartridge loaded with the surgical clips of FIG. 8 and attached to the forceps of FIG. 8, and further showing the surgical clip applier of FIG. 8 aligned with a selected one of the surgical clips;

FIG. 14F depicts a side elevational view of the surgical clip cartridge of FIG. 8, showing the surgical clip cartridge loaded with the surgical clips of FIG. 8 and attached to the forceps of FIG. 8, and further showing the surgical clip applier of FIG. 8 gripping the selected one of the surgical clips;

FIG. 14G depicts a side elevational view of the surgical clip cartridge of FIG. 8, showing the surgical clip cartridge loaded with the surgical clips of FIG. 8 and attached to the forceps of FIG. 8, and further showing the surgical clip applier of FIG. 8 removing the selected one of the surgical clips from the surgical clip cartridge;

FIG. 15 depicts a perspective view of another example of a surgical clip cartridge, showing a magazine of the surgical clip cartridge in a radially expanded state;

FIG. 16 depicts a bottom plan view of the surgical clip cartridge of FIG. 15, showing the magazine in the radially expanded state;

FIG. 17A depicts a front elevational view of the surgical clip cartridge of FIG. 15, showing the magazine in the radially expanded state;

FIG. 17B depicts a front elevational view of the surgical clip cartridge of FIG. 15, showing the magazine in a radially compressed state;

FIG. 18 depicts a partial perspective view of the surgical clip cartridge of FIG. 15, showing the surgical clip cartridge attached to the forceps of FIG. 8 in a first configuration; and

FIG. 19 depicts a partial perspective view of the surgical clip cartridge of FIG. 15, showing the surgical clip cartridge attached to the forceps of FIG. 8 in a second configuration.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. Example of Robotically-Enabled Medical System

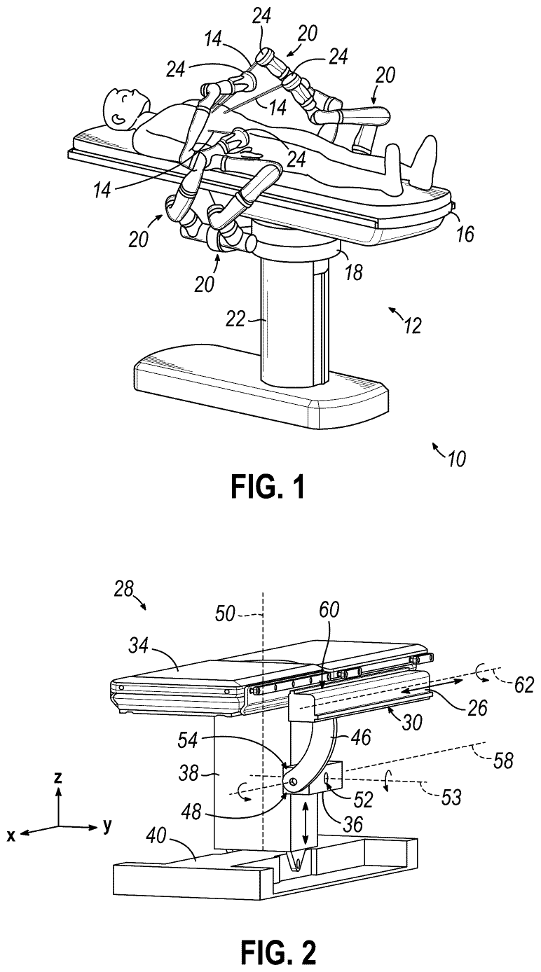

FIG. 1 shows an example of a robotically-enabled medical system, including a first example of a robotic system (10). Robotic system (10) of the present example includes a table system (12) operatively connected to a surgical instrument (14) for a diagnostic and/or therapeutic procedure in the course of treating a patient. Such procedures may include, but are not limited, to bronchoscopy, ureteroscopy, a vascular procedure, and a laparoscopic procedure. To this end, surgical instrument (14) is configured for a laparoscopic procedure, although it will be appreciated that any instrument for treating a patient may be similarly used. At least part of robotic system (10) may be constructed and operable in accordance with at least some of the teachings of any of the various patents, patent application publications, and patent applications that are cited herein.

A. Example of Robotic System with Annular Carriage

As shown in FIG. 1, robotic system (10) includes table system (12) having a platform, such as a table (16), with a plurality of carriages (18) which may also be referred to herein as “arm supports,” respectively supporting the deployment of a plurality of robotic arms (20). Robotic system (10) further includes a support structure, such as a column (22), for supporting table (16) over the floor. Table (16) may also be configured to tilt to a desired angle during use, such as during laparoscopic procedures. Each robotic arm (20) includes an instrument driver (24) configured to removably connect to and manipulate surgical instrument (14) for use. In alternative examples, instrument drivers (24) may be collectively positioned in a linear arrangement to support the instrument extending therebetween along a “virtual rail” that may be repositioned in space by manipulating the one or more robotic arms (20) into one or more angles and/or positions. In practice, a C-arm (not shown) may be positioned over the patient for providing fluoroscopic imaging.

In the present example, column (22) includes carriages (18) arranged in a ring-shaped form to respectively support one or more robotic arms (20) for use. Carriages (18) may translate along column (22) and/or rotate about column (22) as driven by a mechanical motor (not shown) positioned within column (22) in order to provide robotic arms (20) with access to multiples sides of table (16), such as, for example, both sides of the patient. Rotation and translation of carriages (18) allows for alignment of instruments, such as surgical instrument (14), into different access points on the patient. In alternative examples, such as those discussed below in greater detail, robotic system (10) may include a surgical bed with adjustable arm supports including a bar (26) (see FIG. 2) extending alongside. One or more robotic arms (20) may be attached to carriages (18) (e.g., via a shoulder with an elbow joint). Robotic arms (20) are vertically adjustable so as to be stowed compactly beneath table (16), and subsequently raised during use.

Robotic system (10) may also include a tower (not shown) that divides the functionality of robotic system (10) between table (16) and the tower to reduce the form factor and bulk of table (16). To this end, the tower may provide a variety of support functionalities to table (16), such as computing and control capabilities, power, fluidics, optical processing, and/or sensor data processing. The tower may also be movable so as to be positioned away from the patient to improve medical professional access and de-clutter the operating room. The tower may also include a master controller or console that provides both a user interface for operator input, such as keyboard and/or pendant, as well as a display screen, including a touchscreen, for pre-operative and intra-operative information, including, but not limited to, real-time imaging, navigation, and tracking information. In some versions, the tower may include gas tanks to be used for insufflation.

### B. Example of Robotic System with Bar Carriage

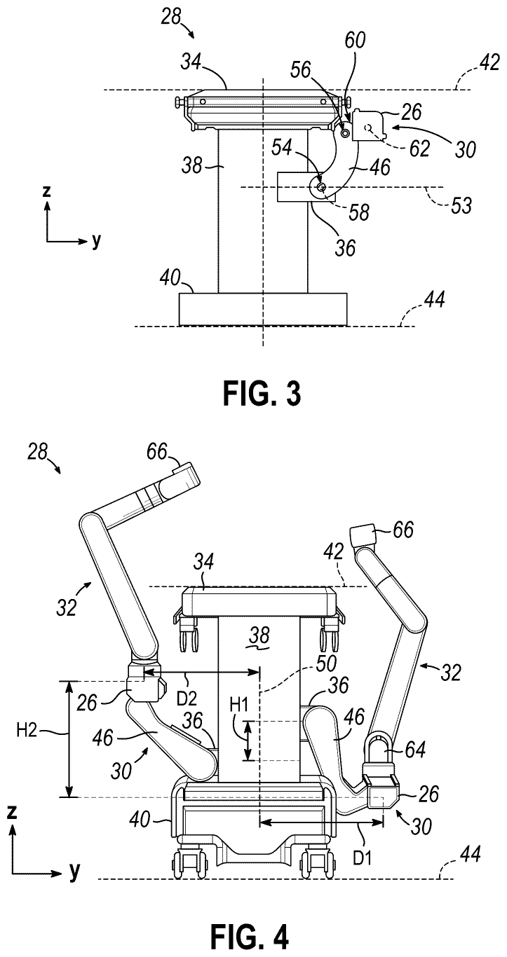

FIGS. 2-4 show another example of a robotic system (28). Robotic system (28) of this example includes one or more adjustable arm supports (30) including bars (26) that are configured to support one or more robotic arms (32) relative to a table (34). In the present example, a single adjustable arm support (30) (FIGS. 2-3) and a pair of adjustable arm supports (30) (FIG. 4) are shown, though additional arm supports (30) may be provided about table (34). Each adjustable arm support (30) is configured to selectively move relative to table (34) so as to alter the position of adjustable arm support (30), and/or any robotic arms (32) mounted thereto, relative to table (34) as desired. Such adjustable arm supports (30) may provide high versatility to robotic system (28), including the ability to easily stow one or more adjustable arm supports (30) with robotic arms (32) beneath table (34).

Each adjustable arm support (30) provides several degrees of freedom, including lift, lateral translation, tilt, etc. In the present example shown in FIGS. 2-4, arm support (30) is configured with four degrees of freedom, which are illustrated with arrows. A first degree of freedom allows adjustable arm support (30) to move in the z-direction (“Z-lift”). For example, adjustable arm support (30) includes a vertical carriage (36). Vertical carriage (36) is configured to move up or down along or relative to a column (38) and a base (40), both of which support table (34). A second degree of freedom allows adjustable arm support (30) to tilt about an axis extending in the y-direction. For example, adjustable arm support (30) includes a rotary joint, which allows adjustable arm support (30) to align with table (34) when table (34) is in a Trendelenburg position or other inclined position. A third degree of freedom allows adjustable arm support (30) to “pivot up” about an axis extending in the x-direction, which may be useful to adjust a distance between a side of table (34) and adjustable arm support (30). A fourth degree of freedom allows translation of adjustable arm support (30) along a longitudinal length of table (34), which extends along the x-direction. Base (40) and column (38) together support table (34) relative to a support surface, which is shown along a support axis (42) above a floor axis (44) in the present example. While the present example shows adjustable arm support (30) mounted to column (38), arm support (30) may alternatively be mounted to table (34) or base (40).

As shown in the present example, adjustable arm support (30) includes vertical carriage (36), a bar connector (46), and bar (26). To this end, vertical carriage (36) attaches to column (38) by a first joint (48), which allows vertical carriage (36) to move relative to column (38) (e.g., such as up and down a first, vertical axis (50) extending in the z-direction). First joint (48) provides the first degree of freedom (“Z-lift”) to adjustable arm support (30). Adjustable arm support (30) further includes a second joint (52), which provides the second degree of freedom (tilt) for adjustable arm support (30) to pivot about a second axis (53) extending in the y-direction. Adjustable arm support (30) also includes a third joint (54), which provides the third degree of freedom (“pivot up”) for adjustable arm support (30) about a third axis (58) extending in the x-direction. Furthermore, an additional joint (56) mechanically constrains third joint (54) to maintain a desired orientation of bar (26) as bar connector (46) rotates about third axis (58). Adjustable arm support (30) includes a fourth joint (60) to provide a fourth degree of freedom (translation) for adjustable arm support (30) along a fourth axis (62) extending in the x-direction.

FIG. 4 shows a version of robotic system (28) with two adjustable arm supports (30) mounted on opposite sides of table (34). A first robotic arm (32) is attached to one such bar (26) of first adjustable arm support (30). This first robotic arm (32) includes a connecting portion (64) attached to a first bar (26). Similarly, a second robotic arm (32) includes connecting portion (64) attached to the other bar (26). As shown in FIG. 4, vertical carriages (36) are separated by a first height (H1), and bar (26) is disposed a second height (H2) from base (40). The first bar (26) is disposed a first distance (D1) from vertical axis (50), and the other bar (26) is disposed a second distance (D2) from vertical axis (50). Distal ends of first and second robotic arms (32) respectively include instrument drivers (66), which are configured to attach to one or more instruments such as those discussed below in greater detail.

In some versions, one or more of robotic arms (32) has seven or more degrees of freedom. In some other versions, one or more robotic arms (32) has eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and connecting portion (64) (1-degree of freedom including translation). In some versions, the insertion degree of freedom is provided by robotic arm (32); while in some other versions, an instrument such as surgical instrument includes an instrument-based insertion architecture.

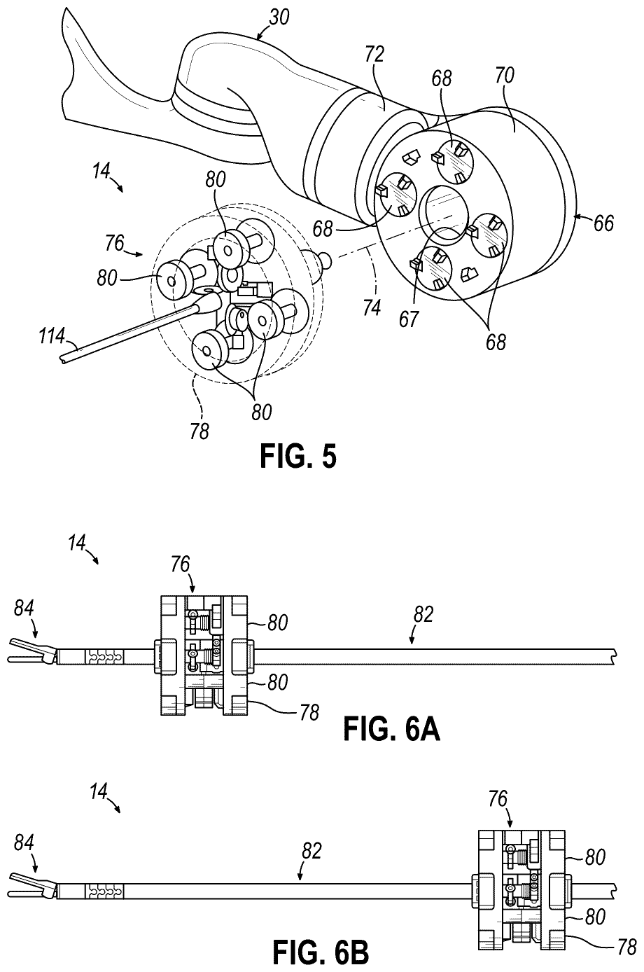

FIG. 5 shows one example of instrument driver (66) in greater detail, with surgical instrument (14) removed therefrom. Given the present instrument-based insertion architecture shown with reference to surgical instrument (14), instrument driver (66) further includes a clearance bore (67) extending entirely therethrough so as to movably receive a portion of surgical instrument (14) as discussed below in greater detail. Instrument driver (66) may also be referred to herein as an “instrument drive mechanism,” an “instrument device manipulator,” or an “advanced device manipulator” (ADM). Instruments may be configured to be detached, removed, and interchanged from instrument driver (66) for individual sterilization or disposal by the medical professional or associated staff. In some scenarios, instrument drivers (66) may be draped for protection and thus may not need to be changed or sterilized.

Each instrument driver (66) operates independently of other instrument drivers (66) and includes a plurality of rotary drive outputs (68), such as four drive outputs (68), also independently driven relative to each other for directing operation of surgical instrument (14). Instrument driver (66) and surgical instrument (14) of the present example are aligned such that the axes of each drive output (68) are parallel to the axis of surgical instrument (14). In use, control circuitry (not shown) receives a control signal, transmits motor signals to desired motors (not shown), compares resulting motor speed as measured by respective encoders (not shown) with desired speeds, and modulates motor signals to generate desired torque at one or more drive outputs (68).

In the present example, instrument driver (66) is circular with respective drive outputs (68) housed in a rotational assembly (70). In response to torque, rotational assembly (70) rotates along a circular bearing (not shown) that connects rotational assembly (70) to a non-rotational portion (72) of instrument driver (66). Power and controls signals may be communicated from non-rotational portion (72) of instrument driver (66) to rotational assembly (70) through electrical contacts therebetween, such as a brushed slip ring connection (not shown). In one example, rotational assembly (70) may be responsive to a separate drive output (not shown) integrated into non-rotatable portion (72), and thus not in parallel to the other drive outputs (68). In any case, rotational assembly (70) allows instrument driver (66) to rotate rotational assembly (70) and drive outputs (68) in conjunction with surgical instrument (14) as a single unit around an instrument driver axis (74).

C. Example of Surgical Instrument with Instrument-based Insertion Architecture

FIGS. 5-6B show surgical instrument (14) having the instrument-based insertion architecture as discussed above. Surgical instrument (14) includes an elongated shaft assembly (82), an end effector (84) connected to and extending distally from shaft assembly (82), and an instrument base (76) (shown with a transparent external skin for discussion purposes) coupled to shaft assembly (82). Instrument base (76) includes an attachment surface (78) and a plurality of drive inputs (80) (such as receptacles, pulleys, and spools) configured to receive and couple with respective rotary drive outputs (68) of instrument driver (66). Insertion of shaft assembly (82) is grounded at instrument base (76) such that end effector (84) is configured to selectively move longitudinally from a retracted position (FIG. 6A) to an extended position (FIG. 6B), vice versa, and any desired longitudinal position therebetween. As used herein, the retracted position is shown in FIG. 6A and places end effector (84) relatively close and proximally toward instrument base (76); whereas the extended position is shown in FIG. 6B and places end effector (84) relatively far and distally away from instrument base (76). Insertion into and withdrawal of end effector (84) relative to the patient may thus be facilitated by surgical instrument (14), although it will be appreciated that such insertion into and withdrawal may also occur via adjustable arm supports (30) in one or more examples.

When coupled to rotational assembly (70) of instrument driver (66), surgical instrument (14), comprising instrument base (76) and instrument shaft assembly (82), rotates in combination with rotational assembly (70) about the instrument driver axis (74). Since instrument shaft assembly (82) is positioned at the center of instrument base (76), instrument shaft assembly (82) is coaxial with instrument driver axis (74) when attached. Thus, rotation of the rotational assembly (70) causes instrument shaft assembly (82) to rotate about its own longitudinal axis. Moreover, as instrument base (76) rotates with instrument shaft assembly (82), any tendons connected to drive inputs (80) of instrument base (76) are not tangled during rotation. Accordingly, the parallelism of the axes of rotary drive outputs (68), rotary drive inputs (80), and instrument shaft assembly (82) allows for the shaft rotation without tangling any control tendons, and clearance bore (67) provides space for translation of shaft assembly (82) during use.

The foregoing examples of surgical instrument (14) and instrument driver (66) are merely illustrative examples. Robotic arms (32) may interface with different kinds of instruments in any other suitable fashion using any other suitable kinds of interface features. Similarly, different kinds of instruments may be used with robotic arms (32), and such alternative instruments may be configured and operable differently from surgical instrument (14).

In addition to the foregoing, robotic systems (10, 28) may be configured and operable in accordance with at least some of the teachings of U.S. Pat. No. 9,737,371, entitled “Configurable Robotic Surgical System with Virtual Rail and Flexible Endoscope,” issued Aug. 22, 2017, the disclosure of which is incorporated by reference herein, in its entirety; U.S. Pat. No. 10,945,904, entitled “Tilt Mechanisms for Medical Systems and Applications,” issued Mar. 16, 2021, the disclosure of which is incorporated by reference herein, in its entirety; U.S. Pub. No. 2019/0350662, entitled “Controllers for Robotically-Enabled Teleoperated Systems,” published Nov. 21, 2019, the disclosure of which is incorporated by reference herein, in its entirety; U.S. Pub. No. 2020/0085516, entitled “Systems and Methods for Concomitant Medical Procedures,” published Mar. 19, 2020; and/or U.S. Pub. No. 2021/0401527, entitled “Robotic Medical Systems Including User Interfaces with Graphical Representations of User Input Devices,” published Dec. 30, 2021, the disclosure of which is incorporated by reference herein, in its entirety.

### II. Surgical Instrument with End Effector

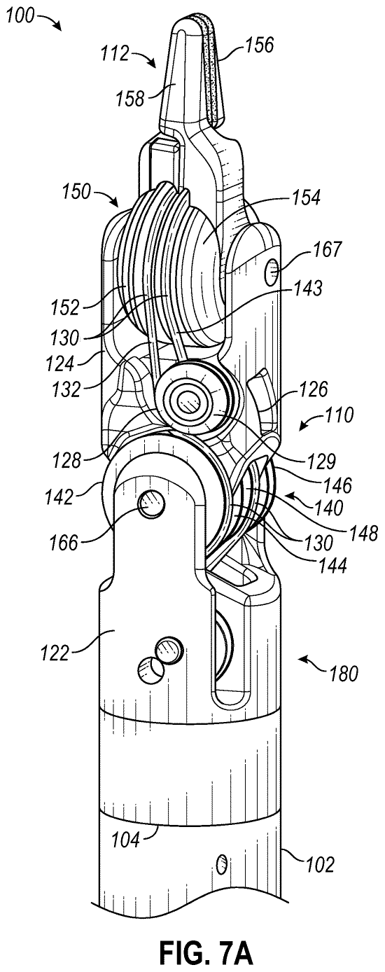

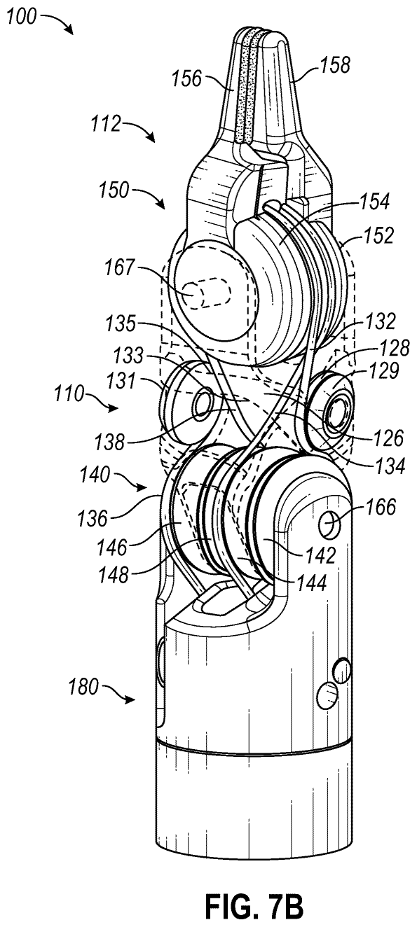

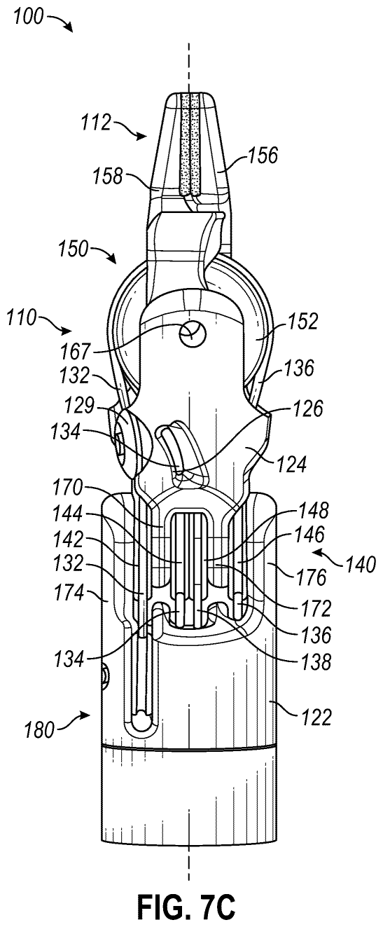

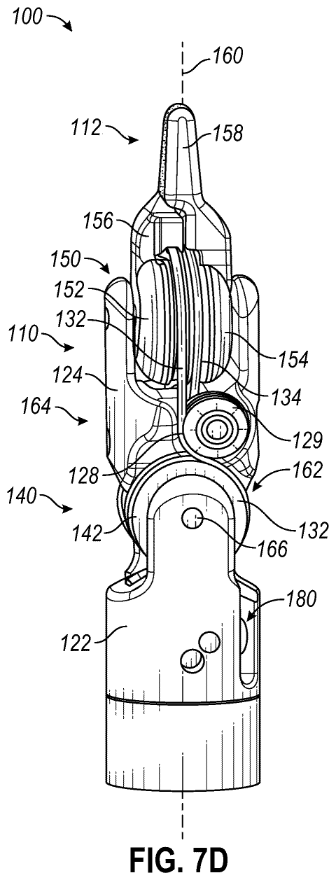

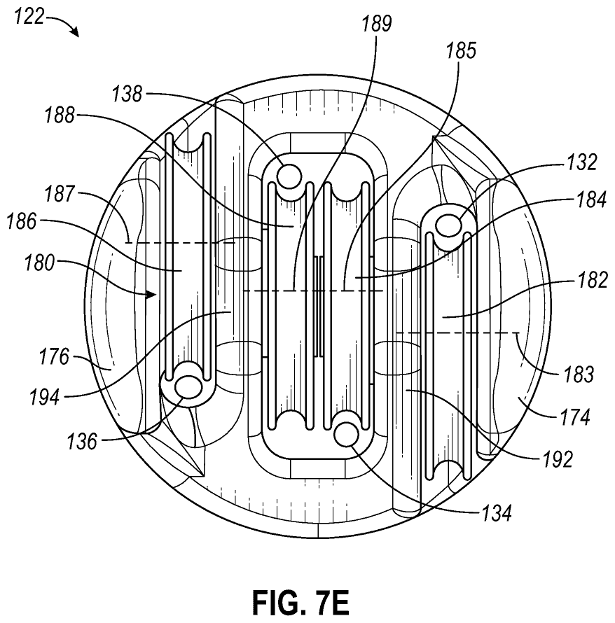

FIGS. 7A-7E illustrate an example of a medical instrument (100) that may be incorporated into a robotic medical system, such as either of the robotic systems (10, 28) described above. For example, instrument (100) may be readily incorporated into either robotic system (10) in place of any of instruments (14).

FIG. 7A is a perspective view of the medical instrument (100). FIG. 7B is another perspective view of the medical instrument (100), shown with a distal clevis (124) illustrated as transparent so as to visualize certain internal features thereof. FIG. 7C is a first side view of the medical instrument (100). FIG. 7D is a second side view of the medical instrument (100). FIG. 7E is a top view of a proximal clevis (122) of the medical instrument (100).

As shown in FIG. 7A, in the illustrated embodiment, the medical instrument (100) includes an elongated shaft (102) extending to a distal end (104). A wrist (110) is positioned at the distal end (104) of the elongated shaft (102). The wrist (110) is also connected to an end effector (112), which is a needle driver in the illustrated embodiment.

In the illustrated embodiment, the wrist (110) comprises a proximal clevis (122) and a distal clevis (124). The proximal clevis (122) can be attached to the distal end (104) of the elongated shaft (102). In the illustrated embodiment, the distal clevis (124) is pivotally attached to the proximal clevis (122) by an axle (166) which extends through the distal clevis (124) and the proximal clevis (122). The distal clevis (124) can rotate about an axis of the axle (166) relative to the proximal clevis (122).

As best seen in FIG. 7C, the proximal clevis (122) can include a first proximal clevis support leg (174) and a second proximal clevis support leg (176). The axle (166) can extend through the first proximal clevis support leg (174) and the second proximal clevis support leg (176) of the proximal clevis (122). Similarly, the distal clevis (124) can include a first distal clevis support leg (170) and a second distal clevis support leg (172). The axle (166) extends through the first distal clevis support leg (170) and the second distal clevis support leg (172) of the distal clevis (124).

As shown in FIGS. 7A-7D, the medical instrument (100) includes a plurality of proximal pulleys (140) and a plurality of distal pulleys (150) positioned in the wrist (110). As best seen in FIGS. 7A-7C, the proximal pulleys (140) can be positioned on the axle (166) that connects the proximal clevis (122) and the distal clevis (124). In the illustrated embodiment, the proximal pulleys (140) include a first outer proximal pulley (142), a first inner proximal pulley (144), a second outer proximal pulley (146), and a second inner proximal pulley (148). The first outer proximal pulley (142), the first inner proximal pulley (144), the second outer proximal pulley (146), and the second inner proximal pulley (148) can each be positioned on the axle (166) such that they can rotate about the axle (166).

As seen in FIGS. 7A-7D, the distal pulleys (150) can be positioned on an axle (167). The axle (167) can extend through the distal clevis (124) as shown. In the illustrated embodiment, the distal pulleys (150) include a first distal pulley (152) and a second distal pulley (154) mounted on the axle (167).

The pitch axle (166) and the yaw axle (167) can be oriented at an angle with respect to each other. In the illustrated example, the pitch axle (166) and the yaw axle (167) are orthogonal. Accordingly, the pitch plane and the yaw plane can also be orthogonal to each other.

The end effector (112) of the medical instrument (100) can be formed by a first jaw member (156) and a second jaw member (158). The first jaw member (156) can be connected to the first distal pulley (152) and the second jaw member (158) can be connected to the second distal pulley (154). The orientation of the end effector (112) can be controlled by rotating the first distal pulley (152) and the second distal pulley (154) in the same direction about the axle (167). For example, by rotating both of the first distal pulley (152) and the second distal pulley (154) in the same direction about the axle (167), the yaw of the end effector (112) can be adjusted. The end effector (112) can be actuated (e.g., opened or closed in the case of the illustrated needle driver) by rotating the first distal pulley (152) and the second distal pulley (154) in the opposite directions about the axle (167).

The medical instrument (100) can include a plurality of pull wires (130) that can be actuated (e.g., pulled or tensioned) to control the three degrees of freedom of the medical instrument (100) (pitch, yaw, and actuation). As shown in FIGS. 7A-7D, the plurality of pull wires (130) are engaged with the proximal pulleys (140) and the distal pulleys (150). In the illustrated embodiment, the plurality of pull wires (130) include a first pull wire segment (132), a second pull wire segment (134), a third pull wire segment (136), and a fourth pull wire segment (138) which are routed along various paths through the wrist (110).

For example, in the illustrated embodiment, the first pull wire segment (132) engages the first outer proximal pulley (142) and the first distal pulley (152). Actuation of the first pull wire segment (132) can be associated with closing the first jaw member (156). The second pull wire segment (134) can be engaged with the first inner proximal pulley (144) and the second distal pulley (154). The second pull wire segment (134) can be associated with opening the second jaw member (158). The third pull wire segment (136) can be engaged with the second outer proximal pulley (146) and second distal pulley (154). The third pull wire segment (136) can be associated with closing the second jaw member (158). The fourth pull wire segment (138) can be engaged with the second inner proximal pulley (148) and the first distal pulley (152). The fourth pull wire segment (138) can be associated with opening the first jaw member (156).

As shown in the figures, each of the first pull wire segment (132) and the fourth pull wire segment (138) can engage the first distal pulley (152), but on opposite sides. Similarly, each of the second pull wire segment (134) and the third pull wire segment (136) can engage the second distal pulley (154), but on opposite sides. In the illustrated embodiment, each of the proximal pulleys (140) is only engaged by one of the pull wire segments. The first pull wire segment (132) engages the first outer proximal pulley (142) on the same side of the wrist (110) that the fourth pull wire segment (138) engages the second inner proximal pulley (148). Similarly, the second pull wire segment (134) engages the first inner proximal pulley (144) on the same side of the wrist (110) that the third pull wire segment (136) engages the second outer, proximal pulley (146). At the proximal pulleys (140), the first and fourth pull wire segments (132, 138) are positioned on an opposite side of the wrist (110) than the second and third pull wire segments (134, 136).

As best seen in FIG. 7B, which illustrates the distal clevis (124) as transparent, the plurality of pull wires (130) are redirected between proximal pulleys (140) and distal pulleys (150). To accomplish the redirection, the wrist (110) of the instrument (100) includes hybrid redirect surfaces. Specifically, in the illustrated embodiment, the wrist (110) includes a pair of static redirect surfaces and a pair of dynamic redirect surfaces positioned between proximal pulleys (140) and distal pulleys (150). As shown in FIG. 7B, the pair of static redirect surfaces include a first static redirect surface (126) and a second static redirect surface (133). The first static redirect surface (126) and the second static redirect surface (133) can each be an angled or curved surface formed in or on the distal clevis (124). An example is visible in FIG. 7C, which shows the static redirect surface (126). The pair of dynamic redirect surfaces include a first dynamic redirect surface (128) and a second dynamic redirect surface (131). Each of the first dynamic redirect surface (128) and the second dynamic redirect surface (131) can comprise a surface of a redirect pulley, such as the first redirect pulley (129) and the second redirect pulley (135) that are illustrated in the figures.

The plurality of pull wires (130) are redirected by the static redirect surfaces (126, 133) and the dynamic redirect surfaces (128, 131). In the illustrated embodiment, the first pull wire segment (132) engages the first dynamic redirect surface (128). The second pull wire segment (134) engages the first static redirect surface (126). The third pull wire segment (136) engages the second dynamic redirect surface (131). The fourth pull wire segment (138) engages the second static redirect surface (133).

Thus, in this example, the first and third pull wire segments (132, 136), which are associated with closing the end effector (112) are redirected using the dynamic redirect surfaces (128, 131) of the redirect pulleys (129, 135), respectively. The second and fourth pull wire segments (134, 138), which are associated with opening the end effector (112) are redirected using the static redirect surfaces (126, 133), respectively.

The medical instrument (100) also includes shaft redirect pulleys (180) positioned in the proximal clevis (122) and/or within the elongated shaft (102). The shaft redirect pulleys (180) are best seen in FIG. 7E which is a top down view of the proximal clevis (122). As shown, the shaft redirect pulleys (180) include a first outer shaft redirect pulley (182), a first inner shaft redirect pulley (184), a second outer shaft redirect pulley (186), and second inner shaft redirect pulley (188). In the illustrated embodiment, the shaft redirect pulleys (180) are in a staggered position. That is, as shown in FIG. 7E, the first outer shaft redirect pulley (182) is positioned on first axis (183) and the first inner shaft redirect pulley (184) is positioned on second axis (185). The first and second axes (183, 185) are not coaxial (in the illustrated embodiment). The second inner shaft redirect pulley (188) is positioned on a third axis (189). In the illustrated embodiment the third axis (189) is coaxial with second axis (185). The second outer shaft redirect pulley (186) is positioned on fourth axis (187). In the illustrated embodiment, the fourth axis (187) is not coaxial with the first, second, or third axes (183, 185, 189). The proximal clevis (122) also comprises a first proximal clevis support wall (192) and a second proximal clevis support wall (194). The first proximal clevis support wall (192) is positioned between the first inner and outer shaft redirect pulleys (182, 184). The second proximal clevis support wall (194) is positioned between the second inner and outer shaft redirect pulleys (186, 184). The first proximal clevis support leg (174) and the second proximal clevis support leg (176) are also shown in FIG. 7E.

By way of further example, medical instrument (100) may be configured and operable in accordance with at least some of the teachings of U.S. Pub. No. 2020/0405423, entitled “Medical Instruments Including Wrists with Hybrid Redirect Surfaces,” published Dec. 31, 2020, the disclosure of which is incorporated by reference herein, in its entirety.

### III. Examples of Surgical Clip Cartridges

In some instances, it may be desirable to provide a clip cartridge that is configured to store a plurality of surgical clips, and that is further configured to be held by a medical instrument, such as medical instrument (100), within a patient. For example, it may be desirable to hold such a clip cartridge via medical instrument (100) within the patient for allowing a surgical clip applier to selectively retrieve one or more of the surgical clips from the clip cartridge while the surgical clip applier is positioned within the patient.

A. Example of Clip Cartridge with Snap-Fit Coupler

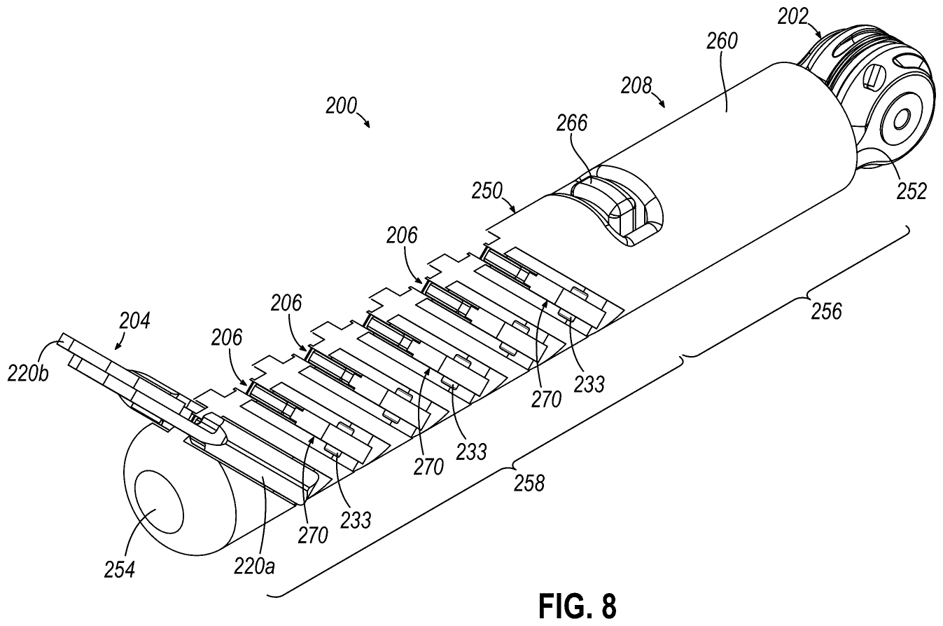

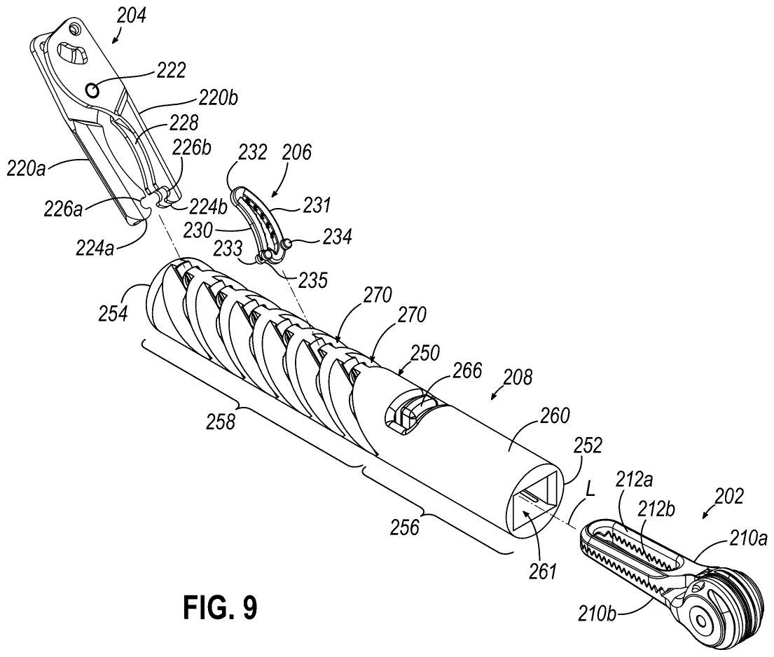

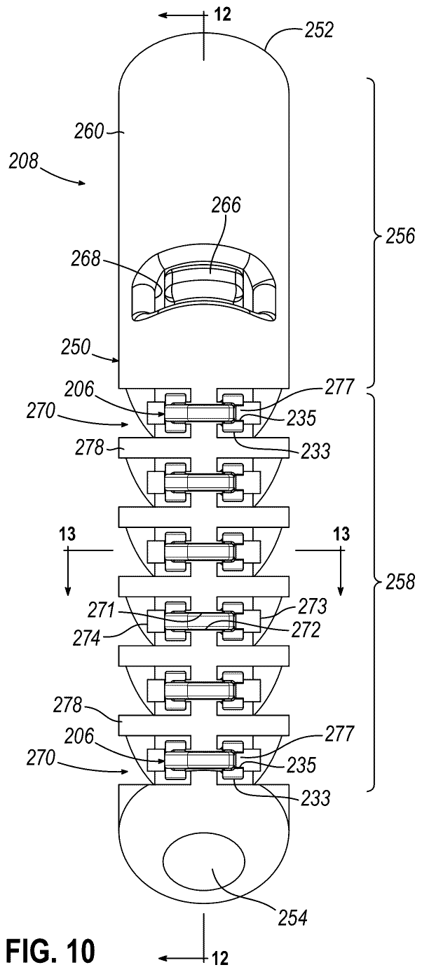

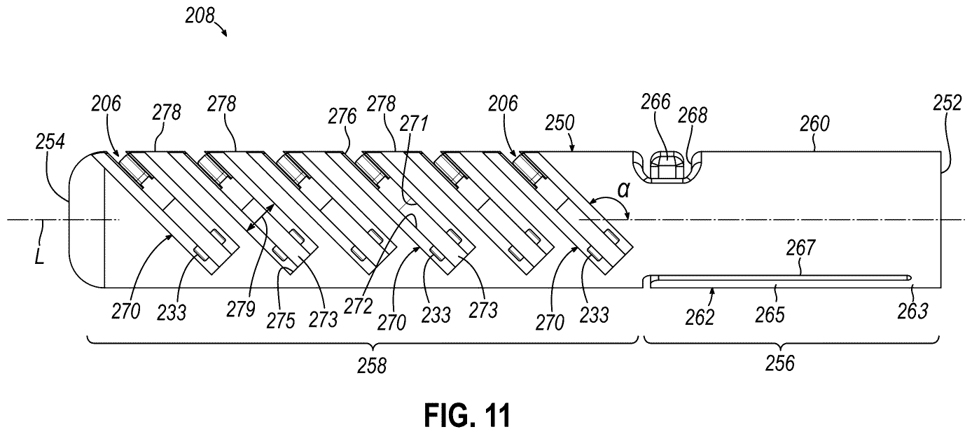

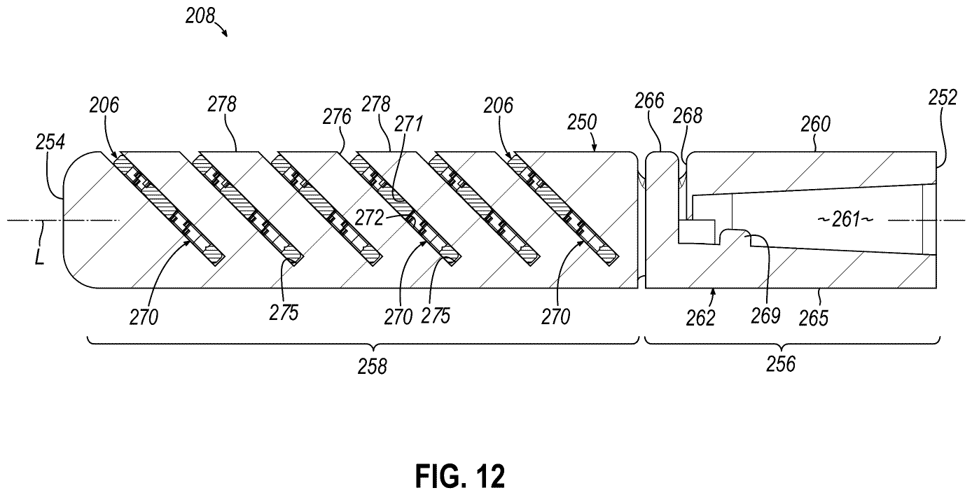

FIGS. 8-14G show an example of surgical system (200) including a first end effector in the form of forceps (202), a second end effector in the form of a surgical clip applier (204), a plurality of surgical clips (206), and a surgical clip cartridge (208) that may provide some, if not all, of the functionalities described above. System (200) may be incorporated into a robotic medical system, such as either of the robotic systems (10, 28) described above. In this regard, forceps (202) and clip applier (204) may be readily incorporated into any of instruments (14, 100) in place of the respective end effectors (84, 112) of such instruments (14, 100). For example, forceps (202) may be readily incorporated into medical instrument (100) in place of end effector (112) described above. In the example shown, forceps (202) include first and second jaw members (210a, 210b) which may be connected to respective distal pulleys (not shown), such that forceps (202) can be actuated (e.g., opened or closed) by rotating the respective distal pulleys in opposite directions in a manner similar to that described above in connection with end effector (112). Thus, forceps (202) may be actuated for manipulating tissue, and/or for holding clip cartridge (208) as described in greater detail below. As shown in FIG. 9, first and second jaw members (210a, 210b) of the present example include first and second elongate apertures (212a, 212b), respectively. By way of further example, forceps (202) may be configured and operable in accordance with at least some teachings of U.S. Pub. No. 2020/0405423, entitled “Medical Instruments Including Wrists with Hybrid Redirect Surfaces,” published Dec. 31, 2020, the disclosure of which is incorporated by reference herein, in its entirety.

In the example shown, clip applier (204) includes first and second jaw members (220a, 220b) that are coupled together at a pivot (222). Each jaw member (220a, 220b) includes a respective distal end (224a, 224b), which are configured to move toward or away from each other based on a closure state of clip applier (204). Each distal end (224a, 224b) includes at least one respective distal engagement feature in the form of a notch (226a, 226b) that is configured to receive a respective portion of a surgical clip (206). In the example shown, each jaw member (220a, 220b) also includes an elongate inner channel (228) that is configured to receive a respective portion of a surgical clip (206). By way of further example, clip applier (204) may be configured and operable in accordance with at least some teachings of International Pub. No. WO 2024/003800, entitled “Safe Mode and Fire Mode for Robotic Clip Applier,” published Jan. 4, 2024, the disclosure of which is incorporated by reference herein, in its entirety.

Each surgical clip (206) of the present example includes a pair of arms (230, 231) that are joined together at a proximal hinge (232); and a pair of pins (233, 234) each extending outwardly from distal regions of arms (230, 231), such that jaws (220a, 220b) are operable to drive each surgical clip (206) from an open state to a closed state by urging pins (233, 234) toward each other. In the example shown, a first pin (233) of each surgical clip (206) is bifurcated by a corresponding slot (235). In some versions, each surgical clip (206) includes a latching feature that is configured to maintain surgical clip (206) in a closed state once surgical clip (206) reaches the closed state. In addition, or in the alternative, each surgical clip (206) may include one or more malleable features that is/are configured to maintain surgical clip (206) in a fully closed state. In some such versions, the one or more malleable features is/are also configured to maintain surgical clip (206) in a fully open state, and/or in a partially closed state, until sufficient force is applied to surgical clip (206) to overcome the malleability. Each surgical clip (206) may comprise any suitable material(s), such as plastic and/or metal. By way of further example, each surgical clip (206) may be configured and operable in accordance with at least some teachings of International Pub. No. WO 2024/003800, entitled “Safe Mode and Fire Mode for Robotic Clip Applier,” published Jan. 4, 2024, the disclosure of which is incorporated by reference herein, in its entirety.

Clip cartridge (208) of the present example includes an elongate body (250) extending along a longitudinal axis (L) from a proximal end (252) to a distal end (254). Body (250) may comprise any suitable material(s), such as plastic and/or metal, and defines a proximal instrument coupler (256) that is configured to facilitate attachment of clip cartridge (208) to forceps (202); and further defines a distal clip magazine (258) that is configured to selectively retain a plurality of surgical clips (206). In the example shown, coupler (256) includes a sleeve (260) that at least partially defines an instrument docking port (261). Docking port (261) extends distally from proximal end (252) toward magazine (258) and is configured to selectively receive forceps (202). In this regard, docking port (261) may be sized and shaped to generally complement the size and shape of forceps (202) in order to provide a close fit between the surfaces of docking port (261) and forceps (202) and thereby inhibit relative movement between forceps (202) and cartridge (208) when forceps (202) are fully received within docking port (261). minimizing an outer dimension (e.g., diameter) of magazine (258). Sleeve (260) may have an outer dimension (e.g., diameter) that is sufficiently small to pass through the working channel of a trocar (T). In some versions, sleeve (260) may have an outer diameter of less than or equal to about 8 mm, such that coupler (256) may be capable of passing through a working channel having a cross dimension (e.g., diameter) of at least about 8 mm. It will be appreciated that sleeve (260) may have any other suitable outer dimension that is sufficiently small to pass through a working channel having any other suitable cross dimension.

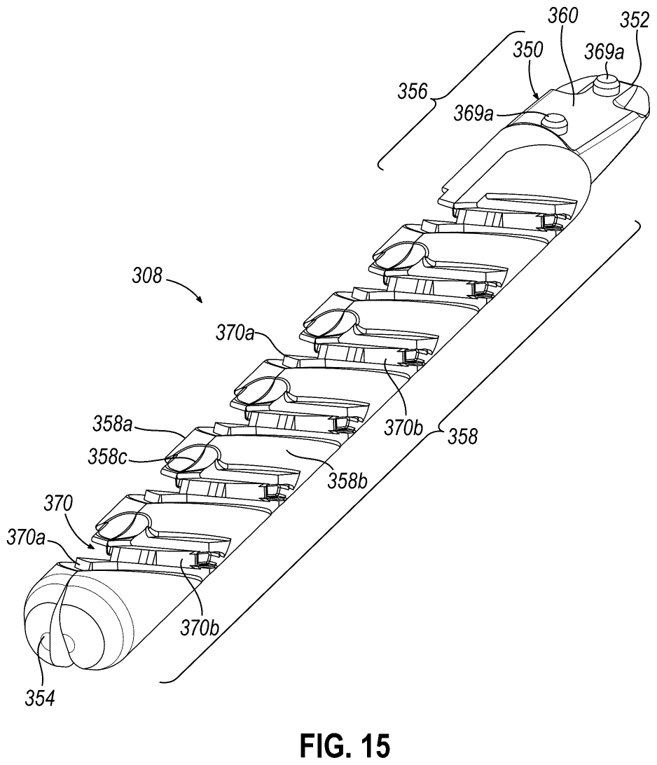

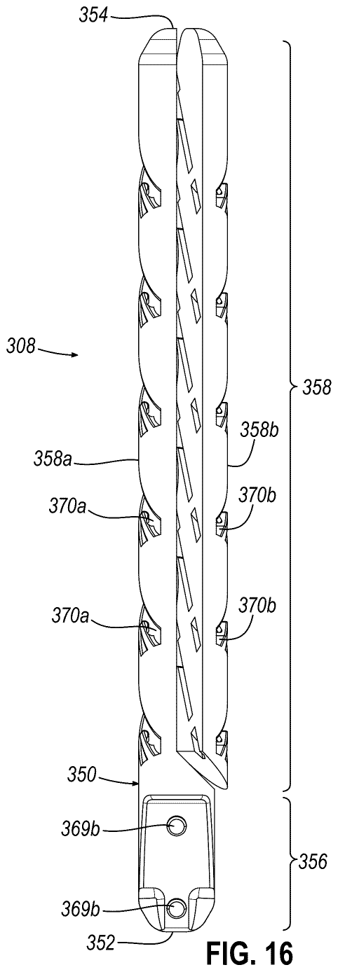

Coupler (256) of the present example further includes a generally L-shaped latch (262) that is cantilevered relative to sleeve (260) at a living hinge (263) such that latch (262) is configured to deflect relative to sleeve (260) about hinge (263). More particularly, latch (262) includes an elongate beam (265) extending distally from hinge (263) along docking port (261), and a button (266) extending upwardly from a distal end of beam (265). In the example shown, beam (265) is defined by a generally U-shaped slot (267) in body (250), and button (266) is exposed through a generally annular opening (268) in body (250), such that latch (262) may be deflected in a downward direction via depressing of button (266). As shown, latch (262) further includes a detent (269) extending upwardly from beam (265) and configured to be selectively received within at least one of apertures (212a, 212b) of forceps (202) when forceps (202) are fully received within docking port (261) for providing a snap-fit engagement between forceps (202) and coupler (256). For example, latch (262) may be resiliently biased toward an undeflected position in which detent (269) may be received within an aperture (212a, 212b) of forceps (202) to secure coupler (256) to forceps (202); and may be deflectable (e.g., via depressing of button (266)) toward a deflected position in which detent (269) may be retracted out of the aperture (212a, 212b) of forceps (202) to release coupler (256) from forceps (202). It will be appreciated that coupler (256) may have any other suitable configuration, such as that described below in connection with FIGS. 15-19.

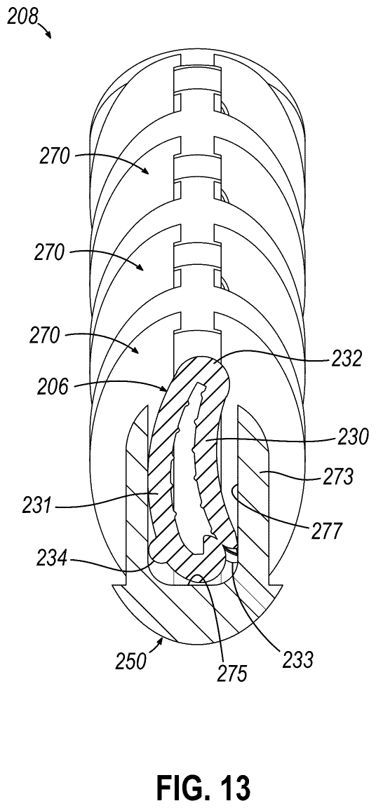

Magazine (258) of the present example includes a longitudinal array of clip receptacles (270) each configured to selectively retain a respective surgical clip (206). Each clip receptacle (270) of the present example is defined by a corresponding proximal wall (271), distal wall (272), pair of side walls (273, 274), closed lower end (275), and open upper end (276). The proximal and distal walls (271, 272) of each clip receptacle (270) are spaced apart from each other to accommodate the arms (230, 231) of the respective surgical clip (206). In some versions, the side walls (273, 274) of each clip receptacle (270) are spaced apart from each other to constrain the respective surgical clip (206) in a partially closed state, which may contribute to frictional engagement between the respective surgical clip (206) and the surfaces of the corresponding clip receptacle (270) (e.g., due to a resilient biasing of each clip (206) toward the open state) and thereby assist with inhibiting inadvertent dislodgement of the respective surgical clip (206) from the corresponding clip receptacle (270). In this regard, each surgical clip (206) may be in a partially closed state when positioned within the corresponding clip receptacle (270), as shown in FIG. 13. In some versions, each surgical clip (206) may be closed to a greater or lesser degree than that depicted in FIG. 13 when positioned within the corresponding clip receptacle (270). In the example shown, a first side wall (273) of each clip receptacle (270) includes a rail (277) that is configured to be received by the slot (235) of the first pin (233) of the respective surgical clip (206). In the example shown, each pair of longitudinally-adjacent clip receptacles (270) are separated from each other by a corresponding partition (278).

Side walls (273, 274) and/or partitions (278) may be configured to accommodate and/or guide clip applier (204) during retrieval of the respective surgical clip (206) from the corresponding clip receptacle (270). For example, side walls (273, 274) and/or partitions (278) may allow guide clip applier (204) to access the respective surgical clip (206) within the corresponding clip receptacle (270), and/or may assist with preventing clip applier (204) from inadvertently contacting another surgical clip (206) within a longitudinally-adjacent clip receptacle (270). In this regard, each pair of longitudinally-adjacent partitions (278) are spaced apart from each other by a corresponding gap (279) that is sized to receive a respective jaw member (220a, 220b) of clip applier (204); and each side wall (273, 274) is sized to be received within an inner channel (228) of the respective jaw member (220a, 220b). Thus, the notch (226a, 226b) of each jaw member (220a, 220b) may at least partially receive a corresponding pin (233, 234) of the respective surgical clip (206) when jaw members (220a, 220b) are received by the corresponding gap (279), with each corresponding side wall (273, 274) received within the inner channels (228) of jaw members (220a, 220b). In this manner, side walls (273, 274) and/or partitions (278) may permit jaws (220a, 220b) to drive each surgical clip (206) toward the closed state by urging pins (233, 234) toward each other and thereby facilitate retrieval of the respective surgical clip (206) from the corresponding clip receptacle (270).

In the example shown, each clip receptacle (270) is oriented obliquely relative to the longitudinal axis (L) of clip cartridge (208). More particularly, the proximal and distal walls (271, 272) of each clip receptacle (270) may be oriented obliquely relative to the longitudinal axis (L) of clip cartridge (208). For example, each clip receptacle (270) may be oriented at an obtuse angle (α) relative to the longitudinal axis (L), such as about 135 degrees. Such an oblique orientation of clip receptacles (270) relative to the longitudinal axis (L) may allow magazine (258) to accommodate surgical clips (206) while minimizing an outer dimension (e.g., diameter) of magazine (258). For example, such an oblique orientation of clip receptacles (270) relative to the longitudinal axis (L) may allow magazine (258) to have an outer dimension (e.g., diameter) that is sufficiently small to pass through the working channel of a trocar (T). In some versions, magazine (258) may have an outer diameter of less than or equal to about 8 mm, such that magazine (258) may be capable of passing through a working channel having a cross dimension (e.g., diameter) of at least about 8 mm. It will be appreciated that magazine (258) may have any other suitable outer dimension that is sufficiently small to pass through a working channel having any other suitable cross dimension. In addition, or alternatively, such an oblique orientation of clip receptacles (270) relative to the longitudinal axis (L) may allow retrieval of surgical clips (206) from clip receptacles (270) by clip applier (204) at a suitable angle of approach, as described in greater detail below.

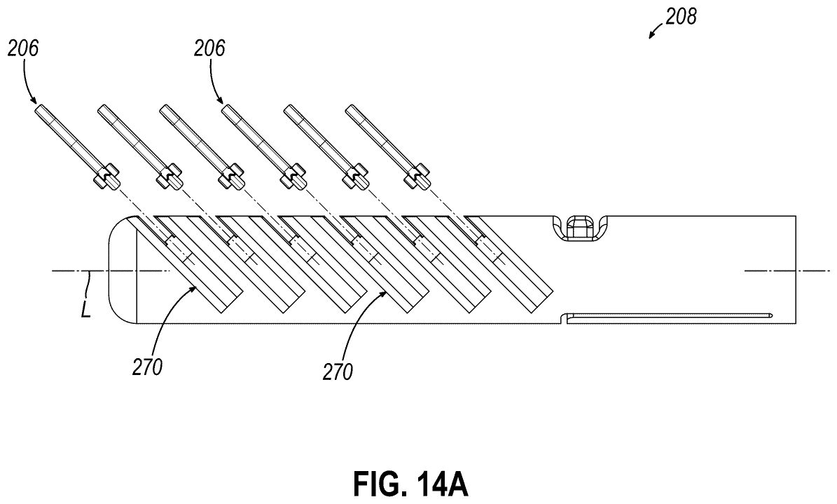

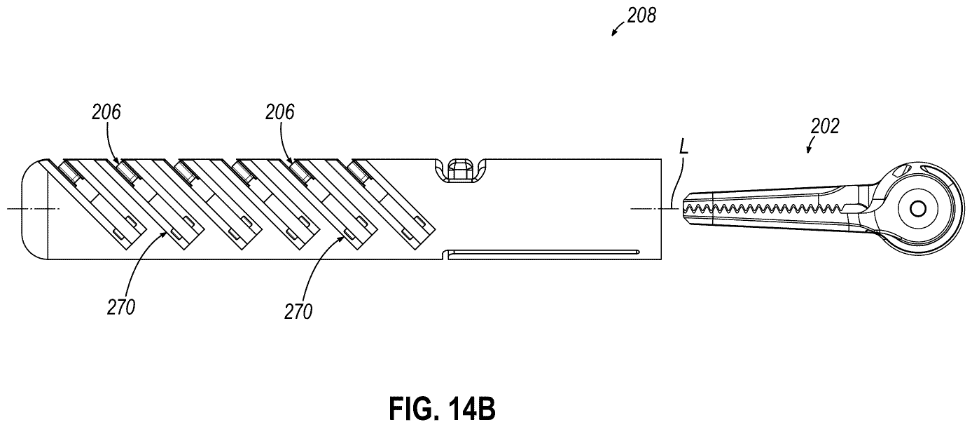

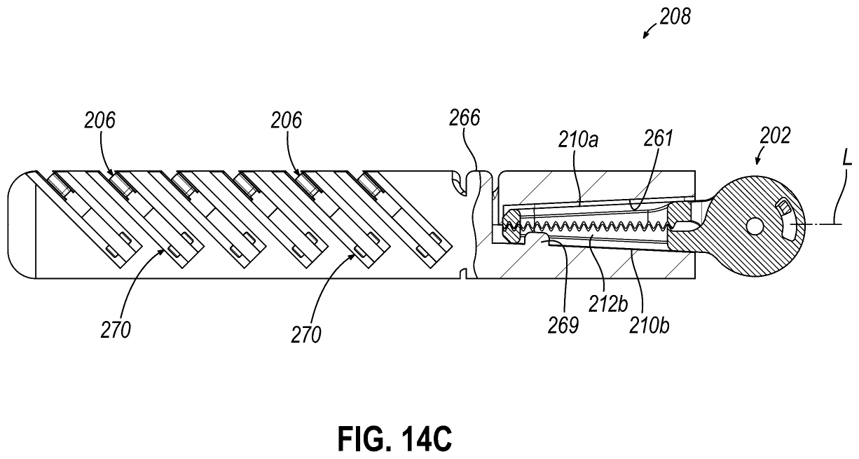

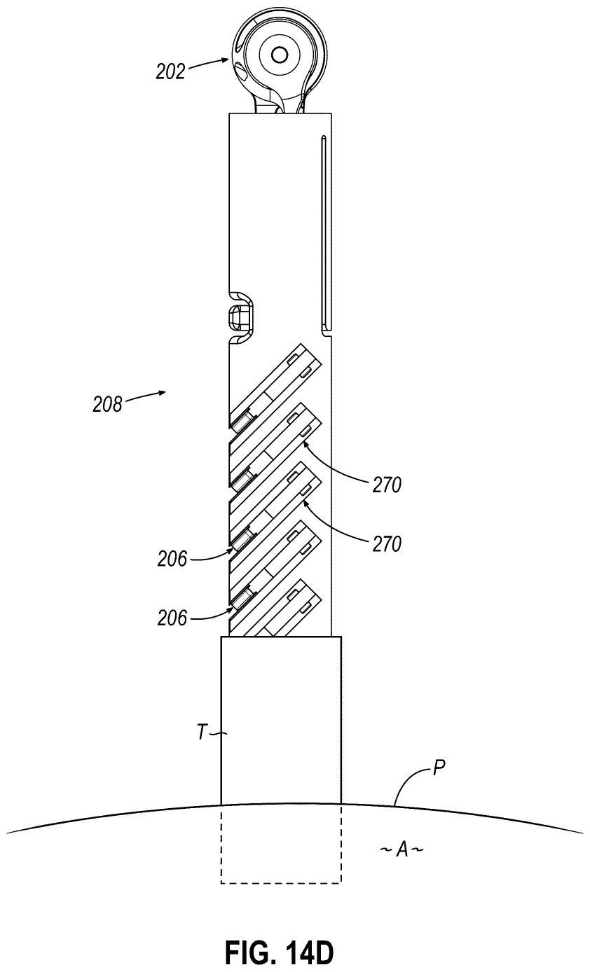

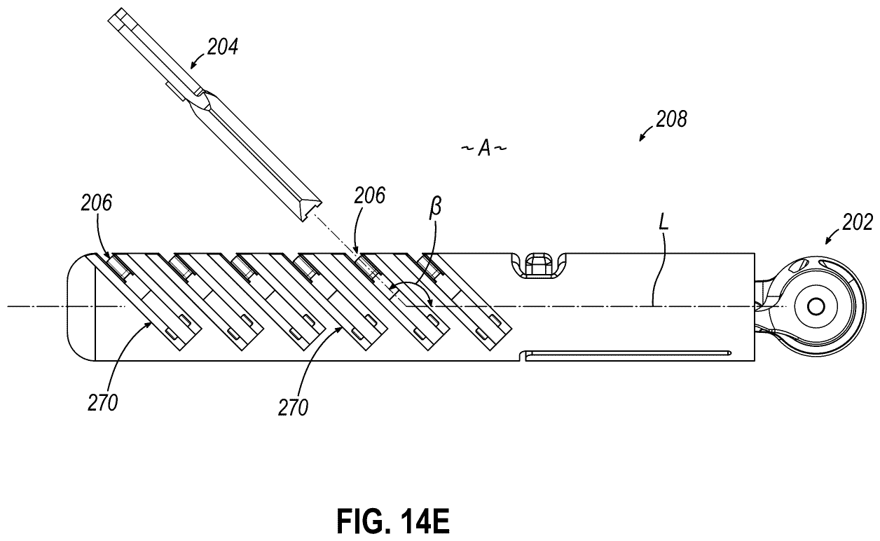

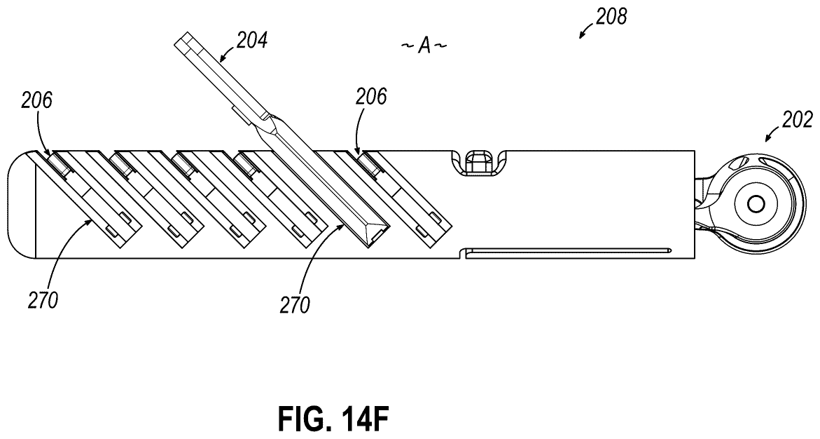

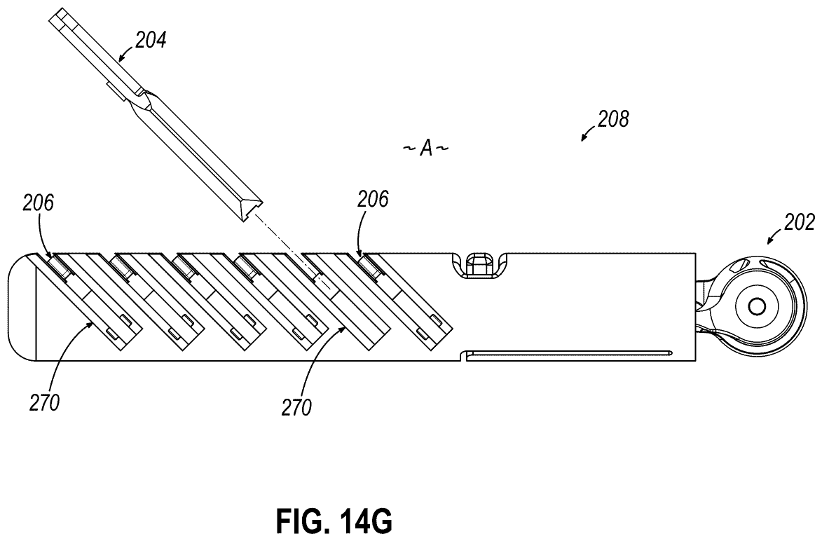

Referring now to FIGS. 14A-14G, in an example of a method of use, each clip receptacle (270) of clip cartridge (208) may initially be empty, and one or more surgical clips (206) may then be loaded into one or more corresponding clip receptacles (270) as shown in FIG. 14A. Such loading of surgical clips (206) into clip receptacles (270) may be performed at any suitable location. For example, surgical clips (206) may be loaded into clip receptacles (270) at a location that is remote from an operating room in which surgical clips (206) will be applied, such as at a manufacturing and/or distribution facility. As another example, surgical clips (206) may be loaded into clip receptacles (270) at or near the operating room in which surgical clips (206) will be applied, such as by a surgeon or surgical technician. As surgical clips (206) are loaded into clip receptacles (270), each surgical clip (206) may frictionally engage one or more surfaces of the corresponding clip receptacle (270) to thereby secure each surgical clip (206) within the corresponding clip receptacle (270). In some instances, each surgical clip (206) may be transitioned from an open state to a partially closed state during loading of surgical clips (206) into clip receptacles (270), and may be constrained by the side walls (273, 274) of the corresponding clip receptacle (270) to remain in the partially closed state, which may contribute to the frictional engagement between the respective surgical clip (206) and the surfaces of the corresponding clip receptacle (270). For example, a resilient biasing of each surgical clip (206) toward the open state may urge the arms (230, 231) and/or pins (233, 234) of each surgical clip (206) outwardly against the side walls (273, 274) and/or rail (277) of the corresponding clip receptacle (270). In addition, or alternatively, the rail (277) of each clip receptacle (270) may be received by the slot (235) of the first pin (233) of the respective surgical clip (206) during such loading, to thereby assist with guiding each surgical clip (206) toward the closed lower end (275) of the corresponding clip receptacle (270); and/or to assist with providing a predetermined orientation of each surgical clip (206) relative to the corresponding clip receptacle (270). In the example shown, since only first pins (233) of surgical clips (206) are bifurcated by corresponding slots (235) for receiving rails (277), each surgical clip (206) may only be loaded into the corresponding clip receptacle (270) when oriented relative to the corresponding clip receptacle (270) such that the respective slot (235) is aligned with the respective rail (277). Thus, rails (277) may serve to inhibit improper loading of surgical clips (206).

With one or more surgical clips (206) loaded into one or more corresponding clip receptacles (270), coupler (256) of clip cartridge (208) may be aligned with forceps (202) for attachment thereto, as shown in FIG. 14B. For example, forceps (202) may be positioned proximally of docking port (261) and aligned therewith along the longitudinal axis (L) of clip cartridge (208). Forceps (202) may then be advanced distally relative to clip cartridge (208) (or clip cartridge (208) may be retracted proximally relative to forceps (202)) such that forceps (202) are inserted into docking port (261) and such that detent (269) is received within at least one of apertures (212a, 212b), as shown in FIG. 14C. In some instances, button (266) may be depressed in a downward direction during insertion of forceps (202) into docking port (261) in order to deflect detent (269) downwardly out of the path of forceps (202); and may then be released to allow detent (269) to resiliently return to the undeflected position such that detent (269) may be received within at least one aperture (212a, 212b) of forceps (202). In addition, or alternatively, a distal end of forceps (202) may cammingly engage detent (269) to urge detent (269) downwardly out of the path of forceps (202); and may then disengage detent (269) to allow detent (269) to resiliently return to the undeflected position such that detent (269) may be received within at least one aperture (212a, 212b) of forceps (202). Thus, coupler (256) of clip cartridge (208) may be secured to forceps (202) via a snap-fit engagement between forceps (202) and coupler (256). While surgical clips (206) are loaded into clip receptacles (270) prior to attachment of clip cartridge (208) to forceps (202) in the present example, it will be appreciated that surgical clips (206) may additionally or alternatively be loaded into clip receptacles (270) after attachment of clip cartridge (208) to forceps (202).

With clip cartridge (208) attached to forceps (202), forceps (202) may be used to insert clip cartridge (208) through a working channel of a trocar (T) and into the body of a patient (P), such as into the patient's abdominal cavity (A) and/or peritoneal cavity, as shown in FIG. 14D. In some instances, the working channel of the trocar (T) may have a cross dimension (e.g., diameter) of at least about 8 mm. It will be appreciated that the working channel of the trocar (T) may have any other suitable cross dimension.

With clip cartridge (208) disposed within the patient's body, clip cartridge (208) may be available to provide intracorporeal loading of one or more surgical clips (206) to clip applier (204), as shown in FIG. 14E. In this regard, clip applier (204) may be inserted into the patient's body, such as into the patient's abdominal cavity and/or peritoneal cavity, via another trocar (not shown). Clip applier (204) may be aligned with one of the clip receptacles (270) of magazine (258) for retrieval of the respective surgical clip (206) therefrom. For example, clip applier (204) may be oriented at an oblique angle of approach (β) relative to the longitudinal axis (L) of clip cartridge (208), which may be substantially equal to the angle (α) at which clip receptacles (270) are oriented relative to the longitudinal axis (L). Thus, the angle of approach (β) may be substantially obtuse. It will be appreciated that such an obtuse angle of approach (β) may assist with facilitating retrieval of surgical clips (206) from clip cartridge (208) by clip applier (204) within substantially confined (e.g., shallow) regions of the patient's body.

Clip applier (204) may then be advanced toward the respective surgical clip (206) along the angle of approach (β), such that jaw members (220a, 220b) of clip applier (204) are received by the respective gaps (279) of the corresponding clip receptacle (270), as shown in FIG. 14F. In some instances, inner channels (228) of jaw members (220a, 220b) may receive the respective side walls (273, 274) of the corresponding clip receptacle (270) during such advancement of clip applier (204). For example, inner channels (228) of jaw members (220a, 220b) may cooperate with the respective side walls (273, 274) of the corresponding clip receptacle (270) to guide such advancement of clip applier (204). Distal ends (224a, 224b) of jaw members (220a, 220b) may then be moved toward each other so that notches (226a, 226b) may receive the respective pins (233, 234) of the respective surgical clip (206). Thus, the selected surgical clip (206) may be gripped by clip applier (204).

With the selected surgical clip (206) gripped by clip applier (204), clip applier (204) may remove the selected surgical clip (206) from clip cartridge (208), as shown in FIG. 14G. In some instances, clip applier (204) may transition the selected surgical clip (206) closer toward a fully closed state prior to removing the selected surgical clip (206) from clip cartridge (208), so as to reduce or eliminate any frictional engagement between the selected surgical clip (206) and the surfaces of the corresponding clip receptacle (270). As shown, clip applier (204) may be retracted together with the selected surgical clip (206) away from clip cartridge (208) along the angle of approach (β).

After removing the selected surgical clip (206) from clip cartridge (208), clip applier (204) may apply the selected surgical clip (206) to a vessel within the patient. Clip applier (204) may then be re-loaded with one or more additional surgical clips (206) from clip applier (204) for application to the same or different vessels within the patient. For example, clip applier (204) may be aligned with another one of the clip receptacles (270) of magazine (258) for retrieval of the respective surgical clip (206) therefrom; advanced toward the respective surgical clip (206) along the angle of approach (β); and used to grip and remove the selected surgical clip (206) from clip cartridge (208). Thus, clip applier (204) may be re-loaded with additional surgical clips (206) from clip cartridge (208) while both clip applier (204) and clip cartridge (208) both remain within the patient.

In some cases, such as when clip cartridge (208) has been emptied of surgical clips (206), forceps (202) may be used to retract clip cartridge (208) through the working channel of trocar (T) and out of the patient's body in order to re-load clip cartridge (208) with one or more additional surgical clips (206). In such cases, clip cartridge (208) may be re-loaded with one or more additional surgical clips (206) while remaining attached to forceps (202), and/or while clip applier (204) remains within the patient. Forceps (202) may then be used to re-insert clip cartridge (208) through the working channel of trocar (T) and into the patient's body for further retrieval of surgical clips (206) from clip cartridge (208) by clip applier (204).

Once the desired number of surgical clips (206) have been retrieved from clip cartridge (208), forceps (202) may be used to retract clip cartridge (208) through the working channel of trocar (T) and out of the patient's body. Coupler (256) of clip cartridge (208) may then be released from forceps (202). For example, latch (262) may be deflected (e.g., via depressing of button (266)) toward the deflected position in which detent (269) is retracted out of the at least one aperture (212a, 212b) of forceps (202); and forceps (202) may be retracted proximally out of docking port (261).

B. Example of Clamshell Clip Cartridge with Clampable Coupler

FIGS. 15-19 show another example of a surgical clip cartridge (308) that may provide some, if not all, of the functionalities described above. Clip cartridge (308) may be similar to clip cartridge (208) described above, except as otherwise described below. In this regard, clip cartridge (308) of the present example includes an elongate body (350) extending along a longitudinal axis from a proximal end (352) to a distal end (354). Body (350) defines a proximal instrument coupler (356) that is configured to facilitate attachment of clip cartridge (308) to forceps (202); and further defines a distal clip magazine (358) that is configured to selectively retain a plurality of surgical clips (206). In the example shown, coupler (356) includes a wedge (360). Wedge (360) narrows toward proximal end (352) and is configured to be selectively clamped by forceps (202).

Coupler (356) of the present example further includes upper and lower pairs of posts (369a, 369b) extending upwardly and downwardly from wedge (360), respectively, and configured to be selectively received within at least one of apertures (212a, 212b) of forceps (202) when forceps (202) are clamped onto wedge (360) for limiting relative movement between forceps (202) and coupler (356). For example, one or both posts (369a, 369b) of each pair may be received within an aperture (212a, 212b) of forceps (202) to secure coupler (356) to forceps (202); and may be released out of the aperture (212a, 212b) of forceps (202) to release coupler (356) from forceps (202). It will be appreciated that coupler (356) may have any other suitable configuration, such as that described above in connection with FIGS. 8-14G.

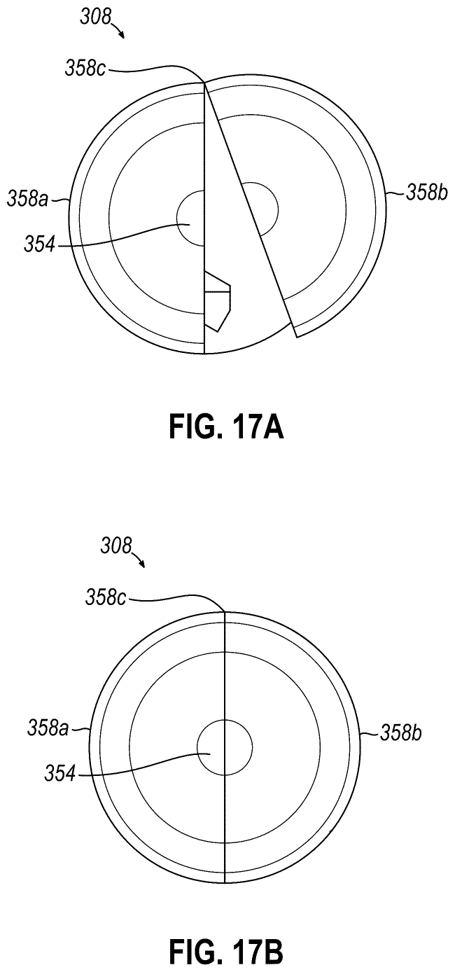

In the example shown, magazine (358) includes first and second longitudinal halves (358a, 358b) pivotably coupled to each other via a longitudinal hinge in the form of a living hinge (358c), such that magazine (358) is configured to transition between a radially expanded state (FIGS. 15, 16, and 17A) and a radially compressed state (FIG. 17B). When in the radially expanded state, magazine (358) may have an outer cross dimension of greater than about 8 mm, such that the radially expanded magazine (358) may not be capable of passing through a working channel having a cross dimension (e.g., diameter) of about 8 mm. When in the radially compressed state, magazine (358) may have an outer diameter of less than or equal to about 8 mm, such that the radially compressed magazine (358) may be capable of passing through a working channel having a cross dimension (e.g., diameter) of at least about 8 mm. It will be appreciated that magazine (358) may have any other suitable outer dimension when in the radially compressed state that is sufficiently small to pass through a working channel having any other suitable cross dimension.

Magazine (358) of the present example includes a longitudinal array of clip receptacles (370) each configured to selectively retain a respective surgical clip (206) in a manner similar to that described above in connection with clip receptacles (370). In the example shown, clip receptacles (370) each straddle both halves (358a, 358b) of magazine (358), such that clip receptacles (370) each include first and second clip receptacle halves (370a, 370b) formed in the first and second longitudinal halves (358a, 358b) of magazine (358), respectively; and such that each clip receptacle (370) is configured to assume a widened state when magazine (358) is in the radially expanded state, and to assume a narrowed state when magazine (358) is in the radially compressed state. When in the widened state, each clip receptacle (370) may have a first width (e.g., defined by the respective side walls) that is sufficiently large to permit the respective surgical clip (206) that is received therein to remain in a fully open state. When in the narrowed state, each clip receptacle (370) may have a second width (e.g., defined by the respective side walls) that is sufficiently small to urge the respective surgical clip (206) that is received therein to a partially closed state, which may contribute to frictional engagement between the respective surgical clip (206) and the surfaces of the corresponding clip receptacle (370) (e.g., due to a resilient biasing of each clip (206) toward the open state) and thereby assist with inhibiting inadvertent dislodgement of the respective surgical clip (206) from the corresponding clip receptacle (370). In this manner, magazine (358) of the present example is configured to reduce or eliminate any potential impact that the constraining of surgical clips (206) within clip receptacles (370) may have on the resilient biasing of surgical clips (206) toward their open states.

In an example of a method of use, surgical clips (206) may be loaded into the corresponding clip receptacles (370) while magazine (358) is in the radially expanded state shown in FIG. 17A, such that clip receptacles (370) are in the widened states. In some cases, magazine (358) may remain in the radially expanded state with clip receptacles (370) in the widened states and loaded with surgical clips (206) for a prolonged period of time before being used in a surgical procedure. For example, surgical clips (206) may be loaded into clip receptacles (370) at a location that is remote from an operating room in which surgical clips (206) will be applied, such as at a manufacturing and/or distribution facility. Clip cartridge (308) may then be packaged, shipped, and/or stored with magazine (358) in the radially expanded state such that clip receptacles (370) remain in the widened states. Thus, the resilient biasing of surgical clips (206) toward their open states may be substantially unaffected by magazine (358) while magazine (358) remains in the radially expanded state.

When clip cartridge (308) is desired for use in a surgical procedure, magazine (358) may be transitioned from the radially expanded state to the radially compressed state, such as to allow magazine (358) to pass through a working channel of a trocar. For example, a surgeon or surgical technician may pivot first and second longitudinal halves (358a, 358b) of magazine (358) toward each other about living hinge (358c) to thereby transition magazine (358) from the radially expanded state to the radially compressed state. As a result, clip receptacles (370) may be transitioned from the widened states to the narrowed states, which may urge the respective surgical clips (206) to their partially closed states.

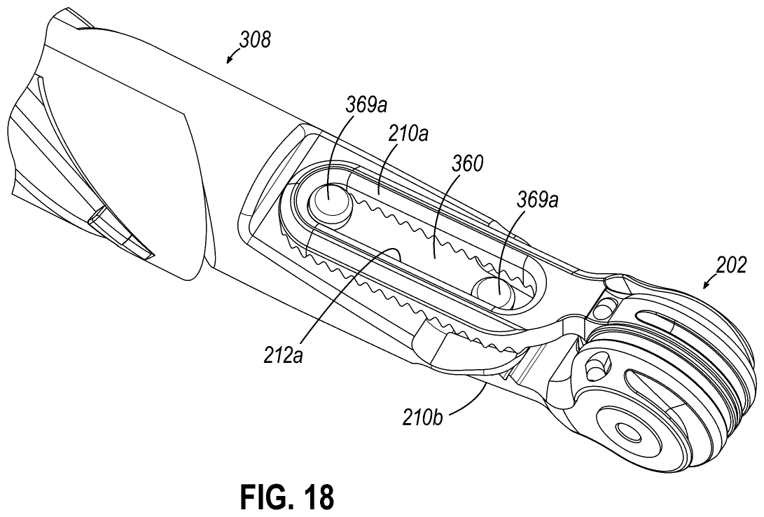

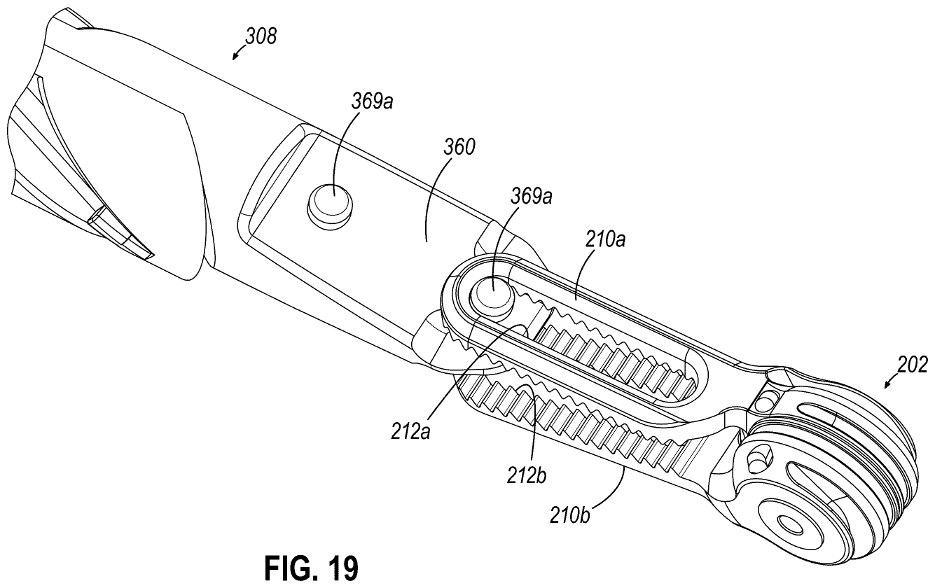

With one or more surgical clips (206) loaded into one or more corresponding clip receptacles (370), and with magazine (358) in the radially compressed state, forceps (202) may be clamped onto wedge (360) such that one or both upper posts (369a) and/or one or both lower posts (369b) are received within respective apertures (212a, 212b) of forceps (202), as shown in FIGS. 18 and 19. For example, both upper posts (369a) and both lower posts (369b) may be received within respective apertures (212a, 212b) of forceps (202), as shown in FIG. 18. In this configuration, clip cartridge (308) may be substantially secured against any movement relative to forceps (202). As another example, only the proximal upper and lower posts (369a, 369b) may be received within respective apertures (212a, 212b) of forceps (202), as shown in FIG. 19. In this configuration, clip cartridge (308) may be pivotable relative to forceps (202) about a pivot axis defined by the proximal upper and lower posts (369a, 369b). It will be appreciated that magazine (358) may be transitioned from the radially expanded state to the radially compressed state before or after attachment of clip cartridge (308) to forceps (202).

With clip cartridge (308) attached to forceps (202), forceps (202) may be used to insert clip cartridge (308) through a working channel of a trocar and into a patient's body, where clip cartridge (308) may be available to provide intracorporeal loading of one or more surgical clips (206) to clip applier (204) in a manner similar to that described above. Since clip cartridge (308) of the present example is attached to forceps (202) via clamping, clip cartridge (308) may be selectively released from forceps (202) while positioned within the patient's body by unclamping forceps (202) from wedge (360). Forceps (202) may then be used to manipulate tissue or perform other operations within the patient's body; and may subsequently be intracorporeally re-clamped onto wedge (360) such that one or both upper posts (369a) and/or one or both lower posts (369b) are received within respective apertures (212a, 212b) of forceps (202), for continued handling of clip cartridge (308) by forceps (202).

### IV. Illustrative Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a proximal instrument coupler configured to releasably attach to an end effector of a surgical instrument; and (b) a distal clip magazine extending distally from the proximal instrument coupler along a longitudinal axis, wherein the distal clip magazine is sized and configured to pass through a working channel of a trocar into a patient's body, wherein the distal clip magazine includes a longitudinal array of clip receptacles, wherein each clip receptacle of the longitudinal array of clip receptacles is configured to selectively retain a respective surgical clip.

### Example 2

The apparatus of Example 1, wherein each clip receptacle of the longitudinal array of clip receptacles is oriented obliquely relative to the longitudinal axis.

### Example 3

The apparatus of Example 2, wherein each clip receptacle of the longitudinal array of clip receptacles is oriented at an angle of about 135 degrees relative to the longitudinal axis.

### Example 4

The apparatus of any of Examples 1 through 3, wherein the distal clip magazine has an outer cross dimension of less than or equal to about 8 mm.

### Example 5

The apparatus of any of Examples 1 through 4, wherein the apparatus comprises an elongate body extending along the longitudinal axis from a proximal end to a distal end, wherein the elongate body defines both the proximal instrument coupler and the distal clip magazine.

### Example 6

The apparatus of any of Examples 1 through 5, wherein the proximal instrument coupler includes an instrument docking port, wherein the instrument docking port is configured to selectively receive the end effector of the surgical instrument.

### Example 7

The apparatus of any of Examples 1 through 6, wherein the proximal instrument coupler includes a deflectable latch, wherein the deflectable latch is configured to provide a snap-fit engagement with a corresponding portion of the end effector of the surgical instrument.

### Example 8

The apparatus of any of Examples 1 through 7, wherein the proximal instrument coupler includes a wedge, wherein the wedge is configured to be clamped by the end effector of the surgical instrument.

### Example 9

The apparatus of any of Examples 1 through 8, wherein the proximal instrument coupler includes at least one post, wherein the at least one post is configured to be captured by a corresponding portion of the end effector of the surgical instrument.

### Example 10

The apparatus of any of Examples 1 through 9, wherein the distal clip magazine is configured to transition between a radially expanded state and a radially compressed state, wherein each clip receptacle of the longitudinal array of clip receptacles is configured to assume a widened state when the distal clip magazine is in the radially expanded state, and wherein each clip receptacle of the longitudinal array of clip receptacles is configured to assume a narrowed state when the distal clip magazine is in the radially compressed state.

### Example 11

A system, comprising: (a) the apparatus of any of Examples 1 through 10; and (b) a plurality of surgical clips, wherein each surgical clip of the plurality of surgical clips is selectively retained within a corresponding clip receptacle of the longitudinal array of clip receptacles of the apparatus.

### Example 12

The system of Example 11, wherein each surgical clip of the plurality of surgical clips frictionally engages at least one surface of the corresponding clip receptacle of the longitudinal array of clip receptacles.

### Example 13

The system of any of Examples 11 through 12, wherein each surgical clip of the plurality of surgical clips is in a partially closed state.

### Example 14

The system of any of Examples 11 through 13, further comprising the end effector of the surgical instrument, wherein the proximal instrument coupler of the apparatus is releasably attached to the end effector.

### Example 15

The system of any of Examples 11 through 14, further comprising a surgical clip applier, wherein the surgical clip applier is configured to selectively remove each surgical clip of the plurality of surgical clips from the corresponding clip receptacle of the longitudinal array of clip receptacles.

### Example 16

A system, comprising: (a) a surgical clip cartridge configured to selectively retain a plurality of surgical clips; and (b) a trocar having a working channel, wherein the surgical clip cartridge is sized and configured to pass through the working channel of the trocar into a patient's body.

### Example 17

A method, comprising: (a) inserting a surgical clip cartridge through a first working channel of a first trocar and into a patient's body, the surgical clip cartridge selectively retaining a plurality of surgical clips; (b) inserting a surgical clip applier through a second working channel of a second trocar and into the patient's body; (c) while the surgical clip cartridge is positioned within the patient's body, removing a first surgical clip of the plurality of surgical clips from the surgical clip cartridge via the surgical clip applier; and (d) applying the first surgical clip of the plurality of surgical clips to an anatomical structure within the patient's body via the surgical clip applier.

### Example 18

The method of Example 17, further comprising removing a second surgical clip of the plurality of surgical clips from the surgical clip cartridge via the surgical clip applier while the surgical clip cartridge remains positioned within the patient's body.

### Example 19

The method of any of Examples 17 through 18, further comprising: (a) attaching the surgical clip cartridge to an end effector of a surgical instrument prior to inserting the surgical clip cartridge through the first working channel of the first trocar and into the patient's body; (b) while the surgical clip cartridge is positioned within the patient's body, releasing the surgical clip cartridge from the end effector of the surgical instrument; and (c) while the surgical clip cartridge remains positioned within the patient's body, re-attaching the surgical clip cartridge to the end effector of the surgical instrument.

### Example 20

The method of any of Examples 17 through 19, further comprising transitioning a clip magazine of the surgical clip cartridge from a radially expanded state to a radially compressed state to thereby urge each surgical clip of the plurality of surgical clips toward a respective closed state prior to inserting the surgical clip cartridge through the first working channel of the first trocar and into the patient's body.

### V. Miscellaneous

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Some versions of the examples described herein may be implemented using a processor, which may be part of a computer system and communicate with a number of peripheral devices via bus subsystem. Versions of the examples described herein that are implemented using a computer system may be implemented using a general-purpose computer that is programmed to perform the methods described herein. Alternatively, versions of the examples described herein that are implemented using a computer system may be implemented using a specific-purpose computer that is constructed with hardware arranged to perform the methods described herein. Versions of the examples described herein may also be implemented using a combination of at least one general-purpose computer and at least one specific-purpose computer.

In versions implemented using a computer system, each processor may include a central processing unit (CPU) of a computer system, a microprocessor, an application-specific integrated circuit (ASIC), other kinds of hardware components, and combinations thereof. A computer system may include more than one type of processor. The peripheral devices of a computer system may include a storage subsystem including, for example, memory devices and a file storage subsystem, user interface input devices, user interface output devices, and a network interface subsystem. The input and output devices may allow user interaction with the computer system. The network interface subsystem may provide an interface to outside networks, including an interface to corresponding interface devices in other computer systems. User interface input devices may include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

In versions implemented using a computer system, a storage subsystem may store programming and data constructs that provide the functionality of some or all of the modules and methods described herein. These software modules may be generally executed by the processor of the computer system alone or in combination with other processors. Memory used in the storage subsystem may include a number of memories including a main random-access memory (RAM) for storage of instructions and data during program execution and a read only memory (ROM) in which fixed instructions are stored. A file storage subsystem may provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem in the storage subsystem, or in other machines accessible by the processor.

In versions implemented using a computer system, the computer system itself may be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the example of the computer system described herein is intended only as a specific example for purposes of illustrating the technology disclosed. Many other configurations of a computer system are possible having more or fewer components than the computer system described herein.

As an article of manufacture, rather than a method, a non-transitory computer readable medium (CRM) may be loaded with program instructions executable by a processor. The program instructions when executed, implement one or more of the computer-implemented methods described above. Alternatively, the program instructions may be loaded on a non-transitory CRM and, when combined with appropriate hardware, become a component of one or more of the computer-implemented systems that practice the methods disclosed.

Versions described above may be designed to be disposed of after a single use, or they can be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof are placed in a closed and sealed container, such as a plastic or TYVEK bag. The container and system, instrument, and/or portion thereof may then be placed in a field of radiation that can penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a proximal instrument coupler configured to releasably attach to an end effector of a surgical instrument; and
(b) a distal clip magazine extending distally from the proximal instrument coupler along a longitudinal axis, wherein the distal clip magazine is sized and configured to pass through a working channel of a trocar into a patient's body, wherein the distal clip magazine includes a longitudinal array of clip receptacles, wherein each clip receptacle of the longitudinal array of clip receptacles is configured to selectively retain a respective surgical clip.

2. The apparatus of claim 1, wherein each clip receptacle of the longitudinal array of clip receptacles is oriented obliquely relative to the longitudinal axis.

3. The apparatus of claim 2, wherein each clip receptacle of the longitudinal array of clip receptacles is oriented at an angle of about 135 degrees relative to the longitudinal axis.

4. The apparatus of claim 1, wherein the distal clip magazine has an outer cross dimension of less than or equal to about 8 mm.

5. The apparatus of claim 1, wherein the apparatus comprises an elongate body extending along the longitudinal axis from a proximal end to a distal end, wherein the elongate body defines both the proximal instrument coupler and the distal clip magazine.

6. The apparatus of claim 1, wherein the proximal instrument coupler includes an instrument docking port, wherein the instrument docking port is configured to selectively receive the end effector of the surgical instrument.

7. The apparatus of claim 1, wherein the proximal instrument coupler includes a deflectable latch, wherein the deflectable latch is configured to provide a snap-fit engagement with a corresponding portion of the end effector of the surgical instrument.

8. The apparatus of claim 1, wherein the proximal instrument coupler includes a wedge, wherein the wedge is configured to be clamped by the end effector of the surgical instrument.

9. The apparatus of claim 1, wherein the proximal instrument coupler includes at least one post, wherein the at least one post is configured to be captured by a corresponding portion of the end effector of the surgical instrument.

10. The apparatus of claim 1, wherein the distal clip magazine is configured to transition between a radially expanded state and a radially compressed state, wherein each clip receptacle of the longitudinal array of clip receptacles is configured to assume a widened state when the distal clip magazine is in the radially expanded state, and wherein each clip receptacle of the longitudinal array of clip receptacles is configured to assume a narrowed state when the distal clip magazine is in the radially compressed state.

11. A system, comprising:
(a) the apparatus of claim 1; and
(b) a plurality of surgical clips, wherein each surgical clip of the plurality of surgical clips is selectively retained within a corresponding clip receptacle of the longitudinal array of clip receptacles of the apparatus.

12. The system of claim 11, wherein each surgical clip of the plurality of surgical clips frictionally engages at least one surface of the corresponding clip receptacle of the longitudinal array of clip receptacles.

13. The system of claim 11, wherein each surgical clip of the plurality of surgical clips is in a partially closed state.

14. The system of claim 11, further comprising the end effector of the surgical instrument, wherein the proximal instrument coupler of the apparatus is releasably attached to the end effector.

15. The system of claim 11, further comprising a surgical clip applier, wherein the surgical clip applier is configured to selectively remove each surgical clip of the plurality of surgical clips from the corresponding clip receptacle of the longitudinal array of clip receptacles.

16. A system, comprising:
(a) a surgical clip cartridge configured to selectively retain a plurality of surgical clips; and
(b) a trocar having a working channel, wherein the surgical clip cartridge is sized and configured to pass through the working channel of the trocar into a patient's body.

17. A method, comprising:
(a) inserting a surgical clip cartridge through a first working channel of a first trocar and into a patient's body, the surgical clip cartridge selectively retaining a plurality of surgical clips;
(b) inserting a surgical clip applier through a second working channel of a second trocar and into the patient's body;
(c) while the surgical clip cartridge is positioned within the patient's body, removing a first surgical clip of the plurality of surgical clips from the surgical clip cartridge via the surgical clip applier; and
(d) applying the first surgical clip of the plurality of surgical clips to an anatomical structure within the patient's body via the surgical clip applier.

18. The method of claim 17, further comprising removing a second surgical clip of the plurality of surgical clips from the surgical clip cartridge via the surgical clip applier while the surgical clip cartridge remains positioned within the patient's body.

19. The method of claim 17, further comprising:
(a) attaching the surgical clip cartridge to an end effector of a surgical instrument prior to inserting the surgical clip cartridge through the first working channel of the first trocar and into the patient's body;
(b) while the surgical clip cartridge is positioned within the patient's body, releasing the surgical clip cartridge from the end effector of the surgical instrument; and
(c) while the surgical clip cartridge remains positioned within the patient's body, re-attaching the surgical clip cartridge to the end effector of the surgical instrument.

20. The method of claim 17, further comprising transitioning a clip magazine of the surgical clip cartridge from a radially expanded state to a radially compressed state to thereby urge each surgical clip of the plurality of surgical clips toward a respective closed state prior to inserting the surgical clip cartridge through the first working channel of the first trocar and into the patient's body.
